(19) United States
(12) Patent Application Publication
(10) Pub. No.: US 2026/0078447 A1
(43) Pub. Date: Mar. 19, 2026

(54) DNA METHYLATION BIOMARKERS OF PREMENSTRUAL DYSPHORIC DISORDER AND PERIMENOPAUSAL DEPRESSION

(71) Applicants: University of Virginia Patent Foundation, Charlottesville, VA (US); University of Ottawa Institute of Mental Health Research (IMHR), Ottawa (CA)

(72) Inventors: Jennifer Payne, Crozet, VA (US); Zachary Kaminsky, Ottawa (CA)

(21) Appl. No.: 19/104,959

(22) PCT Filed: Aug. 22, 2023
(86) PCT No.: PCT/US2023/072636

§ 371 Date: Feb. 19, 2025

Related U.S. Application Data
(60) Provisional application No. 63/373,151, filed on Aug. 22, 2022.

(51) Int. Cl. C12Q 1/6883

(52) U.S. Cl. CPC C12Q 1/6883; C12Q 2600/106; C12Q 2600/118; C12Q 2600/154

(57) ABSTRACT

The present invention relates to the field of Premenstrual dysphoric disorder (PMDD) and perimenopausal depression (PMD). More specifically, the present invention relates to the use of biomarkers to diagnose PMDD, PMD or predict a risk thereof. In one embodiment, a method for identifying a likelihood of PMDD and/or PMD in a patient comprises the steps of (a) providing a sample from the patient; (b) measuring white blood cell type counts and DNA methylation levels of a panel of biomarkers or using a proxy marker to estimate the ratio monocytesmon-monocytes in the sample collected from the patient, wherein the panel of biomarkers comprises HP1BP3 and TTC9B and the white blood cell type counts comprise monocytes and non-monocytes; and (c) using a linear model that utilizes the DNA methylation level of HP1BP3 and TTC9B and the ratio of monocytesmon-monocytes or proxy marker to determine the patient is likely to develop PMDD and/or PMD.

### CROSS-REFERENCE TO RELATED APPLICATIONS

This application claims the benefit of U.S. Provisional Application No. 63/373,151, filed Aug. 22, 2022, the content of which is herein incorporated by reference in its entirety.

### STATEMENT OF GOVERNMENTAL INTEREST

This invention was made with government support under MH112704, MH104262 and MH074799 awarded by the National Institutes of Health. The government has certain rights in the invention.

### INCORPORATION BY REFERENCE OF SEQUENCE LISTING

This application contains a sequence listing. It has been submitted electronically as an XML file titled “2361315.xml.” The sequence listing is 9,996 bytes in size and was created on Aug. 21, 2023. It is hereby incorporated by reference in its entirety.

### FIELD OF THE INVENTION

The present invention relates to the field of reproductive depressions, including premenstrual dysphoric disorder (PMDD) and perimenopausal depression (PMD). More specifically, the present invention relates to the use of biomarkers to diagnose and predict the risk of PMDD and PMD and to provide precision medical treatments for the effective treatments thereof.

### BACKGROUND OF THE INVENTION

Women are twice as likely as men to develop an anxiety or depressive disorder. (Weissman M M, Bland R, Joyce P R, Newman S, Wells J E, Wittchen H U. Sex differences in rates of depression: cross-national perspectives. J Affect Disord. 1993; 29(2-3):77-84). In fact, mood disturbances, related to increased sensitivity to stress triggered by hormone fluctuations in women are common and are poorly addressed. The Australian Bureau of Statistics has estimated that the economic cost of depression and anxiety in women (from lost productivity) could be as high as $22 billion per year (Aust Prescr 2018; 41:183-5 https://doi.org/10.18773/austprescr.2018.060). Moreover, most women do not receive accurate diagnosis, proper care, or the most effective treatments, dramatically impacting their quality of life.

Premenstrual dysphoric disorder (PMDD) is a mood disorder characterized by emotional, cognitive, and physical symptoms. PMDD causes significant distress or impairment in menstruating women during the luteal phase of the menstrual cycle. PMDD has a profound impact on a person's quality of life and dramatically increases the risk of suicidal ideation and even suicide attempts. Despite the high unmet medical need, the broad range of symptoms experienced with PMDD, and their fluctuation in severity, makes an accurate diagnosis of PMDD diagnostically complex requiring at least two months of careful observation.

Accordingly, there is a critical need for new diagnostic approaches to more accurately diagnose PMDD, and to identify the most appropriate precision medical approaches to treat it.

Perimenopausal depression (PMD) is an under-recognized mental health disorder typically affecting women between 42 and 52 years of age, which can have profound effects on a women's health. Women with PMD reported significantly decreased quality of life (QOL), social support, adjustment, suicidal thoughts, and increased disability and compared with non-depressed perimenopausal women (Wariso B A, Guerrieri G M, Thompson K, Koziol D E, Haq N, Martinez P E, et al. Depression during the menopause transition: Impact on quality of life, social adjustment, and disability. Arch Womens Ment Health. (2017) 20:273-82. doi: 10.1007/s00737-016-0701-x). Late-life depression is also an important independent risk factor for vascular dementia (VD) and Alzheimer's disease (AD) (Lin W C, Hu L Y, Tsai S J, Yang A C, Shen C C. Depression and the risk of vascular dementia: a population-based retrospective cohort study. Int J Geriatr Psychiatry. 2017; 32(5): 556-63. Epub 2016 May 11. https://doi.org/10.1002/gps.4493 PMID: 27161941). Additionally previous studies have found that a history of depression increased the risk of dementia twofold (Cherbuin N, Kim S, Anstey K J. Dementia risk estimates associated with measures of depression: a systematic review and meta-analysis. BMJ Open. 2015; 5(12):e008853. Epub 2015 Dec. 23. https://doi.org/10.1136/bmjopen-2015-008853 PMID: 26692556; PubMed Central PMCID: PMC4691713).

The World Health Organization defines the perimenopause as ‘the time immediately preceding the menopause, beginning with endocrine, biologic and clinical changes, and ending a year after the final menstrual period’. The diagnosis of perimenopausal depression is therefore often made retrospectively. To further complicate accurate diagnosis, the physical symptoms of the menopause often present much later (up to five years) than the psychological symptoms. This delay can make the diagnosis of perimenopausal depression very difficult (Willi J, Ehlert U. Assessment of perimenopausal depression: A review. J Affect Disord. (2019) 249:216-22, Maki P M, Kornstein S G, Joffe H, Bromberger J T, Freeman E W, Athappilly G, et al. Guidelines for the evaluation and treatment of perimenopausal depression: Summary and recommendations. J Womens Health (Larchmt). (2019) 28:117-34).

Accordingly, there is an urgent need for better diagnostic approaches which can distinguish PMD from other forms of major depression, and which can be used in a timely fashion to both treat and prevent the development of PMD and to forestall the subsequent development of dementia.

### SUMMARY

Premenstrual dysphoric disorder (PMDD) is considered a type of reproductive affective disorder and is characterized by affective symptoms that emerge in the luteal phase of the menstrual cycle and remit in the follicular phase. Perimenopausal depression (PMD) is an under-recognized mental health disorder which can have profound effects on an older women's health and can lead to an increased risk of dementia. Provided herein it is demonstrated that the disclosed biomarkers can diagnose and predict premenstrual dysphoric disorder (PMDD) and perimenopausal depression (PMD). Further, if a woman develops PMDD or PMD these biomarkers can also be used to determine if she will be responsive to an SSRI. These biomarkers can therefore be used to streamline diagnosis, prevent onset of illness and to identify the best treatment thus improving outcomes.

One aspect provides a method to determine an increased risk or likelihood of developing premenstrual dysphoric disorder (PMDD) and/or perimenopausal depression (PMD), in a patient comprising measuring DNA methylation levels of a panel of biomarker loci in a nucleic acid containing sample from the patient, wherein differential DNA methylation levels of the panel of biomarker loci relative to a corresponding panel of biomarker loci from a patient not having PMDD and/or perimenopausal depression is indicative of the patient having an increased risk of developing PMDD and/or perimenopausal depression, wherein the panel of biomarker loci comprises HP1BP3 and TTC9B.

In one aspect of this method the panel of biomarker loci includes an additional DNA methylation biomarker as a proxy marker to estimate the ratio monocytes:non-monocytes in the sample. In one aspect the additional biomarker loci is MS4A7.

One aspect provides a method to diagnose a patient having one or more symptoms of PMDD or PMD comprising measuring DNA methylation levels of a panel of biomarker loci in a nucleic acid sample obtained from the patient, wherein differential DNA methylation levels of the panel of biomarker loci relative to a corresponding panel of biomarker loci from a patient not having PMDD and/or perimenopausal depression is indicative of the patient having a diagnosis of PMDD and/or perimenopausal depression, wherein the panel of biomarker loci comprises HP1BP3 and TTC9B.

In one aspect of this method the panel of biomarker loci includes an additional DNA methylation biomarker as a proxy marker to estimate the ratio monocytes:non-monocytes in the sample. In one aspect the additional biomarker loci is MS4A7.

Another aspect provides a method to determine an increased risk or likelihood of developing premenstrual dysphoric disorder (PMDD) and/or perimenopausal depression (PMD) in a patient comprising: a) measuring white blood cell type counts and determining a ratio of monocytes:non-monocytes in the sample collected from the patient; or using a proxy marker to estimate the ratio monocytes:non-monocytes in the sample, b) measuring DNA methylation levels of a panel of biomarker loci in a sample collected from the patient, wherein the panel of biomarker loci comprises HP1BP3 loci and TTC9B loci; and c) using a linear model that utilizes the DNA methylation level of HP1BP3 and TTC9B and the ratio of monocytes:non-monocytes or proxy marker to determine the patient is at an increased risk of developing PMDD and/or perimenopausal depression.

In one aspect the ratio of monocytes to non-monocytes is determined by direct measurement through a differential CBC. In one aspect the ratio of monocytes to non-monocytes is determined by measuring intermediate outputs of those cell types like cell surface antigens, or quantifying levels of cell type specific gene products like RNA expression or DNA methylation levels associated with the particular cell types. In one aspect, the ratio of monocytes to non-monocytes may be determined by measuring the DNA methylation status of a monocyte expressed gene which serves as a proxy for this ratio. In one aspect, the monocyte expressed gene is the MS4A locus. In one aspect the biomarker loci is MS4A7.

In one aspect, the patient is a naturally cycling woman, and the sample is collected during the patient's luteal phase. In another aspect the patient is a woman who is taking oral contraceptives, and the method includes the additional step of discontinuing oral contraceptive use by the patient for 3-9 months before the sample is collected in the luteal phase. In another aspect, the methylation levels of the promoter regions of HP1BP3 and TTC9B are measured. In one aspect, HP1BP3 loci comprises CpG dinucleotides located within chr1:20986708-20986650 of human genome build hg 18. In one aspect, the HP1BP3 loci comprises CpG dinucleotides located within the minus strand of chr1:20986708-20986650 of human genome build hg 18. In one aspect, the TTC9B loci comprises CpG dinucleotides located within chr19:45416573 of human genome build hg 18. In one aspect, the TTC9B loci comprises CpG dinucleotides located within the plus strand of chr19:45416573 of human genome build hg 18. In one aspect of this method the methylation levels of MS4A gene cluster loci are used as a proxy the ratio of monocytes:non-monocytes. In one aspect the MS4A loci comprises CpG dinucleotides located within the human MS4A gene cluster on Chromosome 11q12. In one aspect the biomarker loci is MS4A7.

In one aspect, the DNA methylation levels are determined by sodium bisulfite pyrosequencing, methylation-sensitive single nucleotide primer extension (Ms-SNuPE) reaction, methylation-specific PCR or microarray analysis. In one aspect, the DNA methylation levels are measured by amplification using one or more primers comprising SEQ ID NOs: 1-10.

In one aspect the DNA methylation levels are measured after sodium bisulfite modification and analyzed using microarray analysis. In one aspect the microarray analysis is conducted using an Illumina Methyl EPIC microarray assay.

In one aspect, the linear model utilizes DNA methylation at HP1BP3 interacting with the ratio of monocytes:non-monocytes or proxy marker and utilizes DNA methylation at TTC9B as an additive covariate. In another aspect, the linear model utilizes DNA methylation at HP1BP3 and TTC9B as additive covariates and the ratio of monocytes:non-monocytes or proxy marker as an interacting component. In one aspect the linear model uses methylation levels of MS4A gene cluster loci are used as a proxy the ratio of monocytes:non-monocytes. In one aspect the MS4A loci comprises CpG dinucleotides located within the human MS4A gene cluster on Chromosome 11q12. In one aspect the biomarker loci is MS4A7.

In one aspect, the linear model uses a score from Pittsburgh Sleep Quality Index (PSQI) scale taken at the time of sample draw from the patient as an additive or interactive covariate in the model. In one aspect, the linear model uses a score from Clinical Global Impression Scale (CGIS) taken at the time of sample draw from the patient as an additive or interactive covariate in the model. In one aspect, the linear model uses a score from Perceived Stress Scale (PSS) taken at the time of sample draw from the patient as an additive or interactive covariate in the model.

One aspect provides a method of selecting a patient for a PMDD therapy or PMD therapy. In one aspect the PMDD or PMD therapy is anti-depressant therapy. In one aspect the anti-depressant therapy is with an SSRI. In one aspect the PMDD or PMD therapy is hormone replacement therapy. In one aspect the PMDD and PMD therapy is a combination of hormone replacement therapy and anti-depressant therapy. In one aspect the hormone replacement therapy is combined with anti-depressant therapy with an SSRI.

One aspect provides a method to treat a patient responsive to serotonin reuptake inhibitors (SSRIs) and at risk of developing, or having, premenstrual dysphoric disorder (PMDD) and/or perimenopausal depression comprising a) identifying the patient is an SSRI responder, and b) administering to the patient an effective SSRI, wherein the risk of developing premenstrual dysphoric disorder (PMDD) and/or perimenopausal depression and responsiveness to SSRIs is determined by: a) measuring white blood cell type counts and determining a ratio of monocytes:non-monocytes in the sample collected from the patient during the patient's luteal phase or using a proxy marker to estimate the ratio monocytes:non-monocytes in the sample, b) measuring DNA methylation levels of a panel of biomarker loci in the sample collected from the patient, wherein the panel of biomarker loci comprises HP1BP3 loci and TTC9B loci; and c) using a linear model that utilizes the DNA methylation level of HP1BP3 and TTC9B and the ratio of monocytes:non-monocytes, or proxy marker to determine the patient is at an increased risk of developing PMDD and/or perimenopausal depression and if the patient will be responsive to selective serotonin reuptake inhibitors (SSRIs). In one aspect the methylation levels of MS4A gene cluster loci are used as a proxy the ratio of monocytes:non-monocytes. In one aspect the MS4A loci comprises CpG dinucleotides located within the human MS4A gene cluster on Chromosome 11q12. In one aspect the biomarker loci is MS4A7.

Another aspect provides a method to treat a patient at risk of developing premenstrual dysphoric disorder (PMDD) and/or perimenopausal depression comprising administering to the patient an effective anti-depressant, wherein the risk of developing premenstrual dysphoric disorder (PMDD) and/or perimenopausal depression is determined by: a) measuring white blood cell type counts and determining a ratio of monocytes:non-monocytes in the sample collected from the patient during the patient's luteal phase or using a proxy marker to estimate the ratio monocytes:non-monocytes in the sample, b) measuring DNA methylation levels of a panel of biomarker loci in the sample collected from the patient, wherein the panel of biomarker loci comprises HP1BP3 loci and TTC9B loci; and c) using a linear model that utilizes the DNA methylation level of HP1BP3 and TTC9B and the ratio of monocytes:non-monocytes or proxy marker to determine the patient is at an increased risk of developing PMDD and/or perimenopausal depression. In one aspect of this method the methylation levels of MS4A gene cluster loci are used as a proxy the ratio of monocytes:non-monocytes. In one aspect the MS4A loci comprises CpG dinucleotides located within the human MS4A gene cluster on Chromosome 11q12. In one aspect the biomarker loci is MS4A7.

One aspect provides a method to determine premenstrual dysphoric disorder (PMDD) and/or perimenopausal depression therapy (PMD) for a patient having or at risk of having PMDD and/or perimenopausal depression therapy comprising: a) measuring DNA methylation levels of a panel of biomarker loci in a nucleic acid containing sample from the patient, wherein differential DNA methylation levels of the panel of biomarker loci relative to a corresponding panel of biomarker loci from a patient not having PMDD and/or perimenopausal depression is indicative of the patient having an increased risk of developing PMDD and/or perimenopausal depression, wherein the panel of biomarker loci comprises HP1BP3 and TTC9B; and a proxy marker for the ratio monocytes:non-monocytes in the sample, and b) using the relative methylation levels of the proxy marker loci, HP1BP3 loci and the TTC9B loci of the patient compared to an appropriate control sample to determine the PMDD or perimenopausal depression therapy. In one aspect the methylation levels of MS4A gene cluster loci are used as the proxy marker for the ratio of monocytes:non-monocytes. In one aspect the MS4A loci comprises CpG dinucleotides located within the human MS4A gene cluster on Chromosome 11q12. In one aspect the biomarker loci is MS4A7.

In one aspect of the methods, the methylation levels of the promoter regions of HP1BP3 and TTC9B are measured. In one aspect of the methods, the HP1BP3 loci comprises CpG dinucleotides located within chr1:20986708-20986650 of human genome build hg 18. In one aspect of the methods, the HP1BP3 loci comprises CpG dinucleotides located within the minus strand of chr1:20986708-20986650 of human genome build hg 18. In one aspect of the methods, the TTC9B loci comprises CpG dinucleotides located within chr19:45416573 of human genome build hg 18. In one aspect of the methods, the TTC9B loci comprises CpG dinucleotides located within the plus strand of chr19:45416573 of human genome build hg 18. In one aspect of this method the methylation levels of MS4A gene cluster loci are used as a proxy the ratio of monocytes:non-monocytes. In one aspect the MS4A loci comprises CpG dinucleotides located within the human MS4A gene cluster on Chromosome 11q12. In one aspect the biomarker loci is MS4A7.

In one aspect of the methods, the DNA methylation levels are determined by sodium bisulfite pyrosequencing, methylation-sensitive single nucleotide primer extension (Ms-SNuPE) reaction, methylation-specific PCR or microarray analysis. In one aspect of the methods, the DNA methylation levels are measured by amplification using one or more primers comprising SEQ ID NOs: 1-10. In one aspect the DNA methylation levels are measured after sodium bisulfite modification and analyzed using microarray analysis. In one aspect the microarray analysis is conducted using an Illumina Methyl EPIC microarray assay.

In one aspect of the methods, the linear model utilizes DNA methylation at HP1BP3 interacting with the ratio of monocytes:non-monocytes or a proxy marker for the ratio monocytes:non-monocytes in the sample, and utilizes DNA methylation at TTC9B as an additive covariate. In one aspect of the methods, the linear model utilizes DNA methylation at HP1BP3 and TTC9B as additive covariates and the ratio of monocytes:non-monocytes or a proxy marker for the ratio monocytes:non-monocytes in the sample, as an interacting component.

In one aspect the linear model uses methylation levels of MS4A gene cluster loci as a proxy the ratio of monocytes:non-monocytes. In one aspect the MS4A loci comprises CpG dinucleotides located within the human MS4A gene cluster on Chromosome 11q12. In one aspect the biomarker loci is MS4A7.

In one aspect of the methods, the linear model uses a score from Pittsburgh Sleep Quality Index (PSQI) scale taken at the time of sample draw from the patient as an additive or interactive covariate in the model. In one aspect of the methods, the linear model uses a score from Clinical Global Impression Scale (CGIS) taken at the time of sample draw from the patient as an additive or interactive covariate in the model. In one aspect of the methods, the linear model uses a score from Perceived Stress Scale (PSS) taken at the time of sample draw from the patient as an additive or interactive covariate in the model.

In one aspect, the anti-depressant is an SSRI. In one aspect, SSRI is citalopram, escitalopram, fluoxetine, fluvoxamine, paroxetine, sertraline, vilazodone or a combination thereof.

One aspect comprises administering the PMMD and/or perimenopausal depression therapy (PMD) to the patient. In one aspect, the PMMD and/or perimenopausal depression therapy is determined using a classification algorithm to determine PMMD and/or perimenopausal depression status using methylation levels from known samples. In one aspect, the classification algorithm uses one or more additives or interactive covariates to determine the PMMD and/or perimenopausal depression therapy of the patient. In one aspect, the classification algorithm uses stress, anxiety, or sleep quality metrics from the patient as an additive or interactive covariate. In one aspect, the stress, anxiety, or sleep quality metrics are taken from Pittsburgh Sleep Quality Scale (PSQI), Clinical Global Impression Scale (CGIS), Perceived Stress Scale (PSS) or a combination thereof. In one aspect, the treatment is psychiatric, Cognitive Behavioral Therapy (CBT), antidepressant medication, hormone therapy or a combination thereof. In one aspect, the antidepressant medication is a selective serotonin reuptake inhibitor (SSRI). In one aspect, the SSRI is citalopram, escitalopram, fluoxetine, fluvoxamine, paroxetine, sertraline, vilazodone or a combination thereof.

One aspect provides a method to monitor the progress of a patient on treatment for premenstrual dysphoric disorder (PMDD) and/or perimenopausal depression comprising: a) measuring DNA methylation levels of a panel of biomarker loci in a nucleic acid containing sample collected from the patient for at least two points during a course of treatment, wherein the panel of biomarker loci comprises HP1BP3 and TTC9B; and b) identifying a change in DNA methylation levels of HP1BP3 and TTC9B from a), wherein the change in the methylation status identified in b) towards normal levels indicates the treatment is therapeutically efficacious for the patient, wherein normal levels are determined by comparison to a suitable control sample. In one aspect, the change in methylation levels is in the promoter region of HP1BP3 and/or TTC9B. In one aspect, the control sample is derived from an unaffected individual or normal control sample. In one aspect of this method the panel of biomarker loci includes an additional DNA methylation biomarker as a proxy marker to estimate the ratio monocytes:non-monocytes in the sample. In one aspect the additional biomarker loci is MS4A. In one aspect the biomarker loci is MS4A7.

One aspect provides a method to select a patient that will respond to SSRI therapy most effectively, comprising: a) measuring DNA methylation levels of a panel of biomarker loci in a sample collected from the patient, wherein the panel of biomarker loci comprises HP1BP3 loci, TTC9B loci and MS4A loci; and c) using a linear model that utilizes the DNA methylation level of HP1BP3 and TTC9B and MS4A7 to determine that the patient will respond to an SSRI.

### BRIEF DESCRIPTION OF THE DRAWINGS

FIG. 1. Boxplots of PPD model prediction outputs for women with PMDD and controls (A), SSRI responders and non-responders (C), and PMDD SSRI responders vs controls (E). Receiver operator characteristic curves depicting the sensitivity (y axis) as a function of specificity (x axis) for the prediction of PMDD status (B), SSRI response status (D), and PMDD status among SSRI responders (F).

FIG. 2. Boxplots of PPD model prediction outputs for women with W6 EPDS scores >13 and those with EPDS <13 (A) and SSRI responders and non-responders (C). Receiver operator characteristic curves depicting the sensitivity (y axis) as a function of specificity (x axis) for the prediction of EPDS score status (B) and SSRI response status (D). Future model outputs will be depicted as probabilities.

FIG. 3. A) Boxplot of the model output (y axis) as a function of depression post menopause (x axis). B.) Receiver operator characteristic curve of the prediction of perimenopausal depression (PMD) using the biomarker model. The predictive accuracy supports the supposition that the model can predict a broad range of reproductive depressions including PMDD and peri-menopausal depression. Future model outputs will be depicted as probabilities.

FIG. 4. A heuristic which represents the generation of SSRI responder status.

### DETAILED DESCRIPTION OF THE INVENTION

As used herein, the terms “Reproductive Subtype of Depression,” or “Reproductive Depression” refer to depressive episodes or symptoms that occur in the setting of normal reproductive hormone fluctuations in women (Payne, et al, 2009). Reproductive depression can be conceptualized as an abnormal brain response to times of normal hormonal change that occur during the reproductive years in women including the premenstrual, postpartum and perimenopausal time-periods. Each of these time-periods carries an increased risk of the development of a major depressive episode and having one type of reproductive depression increases the likelihood that a woman will experience another type of reproductive depression.

Further, the genetic basis for reproductive depressions such as postpartum depression (PPD), perimenopausal depression (PMD), and premenstrual dysphoric disorder (PMDD) appears to be separate from that of Major Depression, indicating that reproductive depressions may have a biological basis that is distinct from that of Major Depression.

Several studies have elegantly demonstrated that it is the change in reproductive hormone levels that trigger the mood symptoms. For example, in a sample of 8 women with a history of PPD, mood symptoms recurred when high doses of estrogen and progesterone were withdrawn, modeling the hormonal changes that occur with birth (Bloch et al, 2000). In contrast, none of the healthy control women who had previously been pregnant but had not experienced PPD experienced mood symptoms. In a similar study, Schmidt et al, first stabilized hormonal fluctuations using the gonadotropin leuprolide and then, in a blinded fashion, added back estrogen or progesterone. Again, only women with a history of PMDD experienced mood symptoms when either estrogen or progesterone was added in contrast to healthy controls. This same sensitivity to hormonal change has also been demonstrated in perimenopausal depression (Daly, 2003). Thus, it is the change in reproductive hormone levels that leads to reproductive depression, indicating that the biological basis is likely to be different than that for Major Depression that is not triggered by hormonal change.

There is some evidence that at least some reproductive depressions may be uniquely and rapidly responsive to selective serotonin reuptake inhibitors (SSRIs). First, several studies have suggested that women respond better to SSRI's during their reproductive years than postmenopausal women whose hormonal fluctuations have stabilized (reviewed in Payne, 2009). Further, approximately 50% of women with PMDD respond to luteal (premenstrual) phase treatment with SSRI's and this treatment response is rapid (within hours or days), thus indicating a different mechanism of action than that of SSRI use in Major Depression which takes weeks to months to achieve response. Though not well studied, the literature on the treatment of PPD also indicates a potentially superior, and possibly more rapid response to SSRI in some patients. There have been two small, randomized clinical trials of PPD recurrence prevention and one in the treatment of PPD. The SSRI sertraline prevented recurrence in more women compared to placebo while the tricyclic antidepressant nortriptyline did not (Wisner 2004). In a randomized trial comparing sertraline and nortriptyline in the treatment of PPD, the response to sertraline was significant within the first week of treatment but ultimately no different from the response rate to nortriptyline (Wisner 2006). Therefore, at least some reproductive subtypes of depression are uniquely and rapidly responsive to SSRI treatment and there may be important pathophysiological differences between reproductive depressions that are responsive to SSRI's compared to those that are not. Developing a reliable bioassay capable of distinguishing SSRI responsive forms of reproductive depressions therefore offers the door to more both more accurate diagnosis and more effective precision medical treatments with the most effective anti-depressants.

Previously, we have identified a panel of epigenetic biomarkers based on the TTC9B and HP1BP3 genes that are predictive of postpartum depression with approximately 80% accuracy when sampled in the third trimester when reproductive hormones levels are elevated (US 2022/0049304; U.S. Pat. No. 10,865,446 both of which are incorporated herein by reference). In the current study we have extended these original discoveries and determined the same biomarkers can also be modelled to predict premenstrual dysphoric disorder (PMDD) and perimenopausal depression. Additionally, we show that here for the first time that the MS4A loci gene locus can be used as an effective approach to normalize the ratio of monocytes to non-monocytes to enhance the precision and sensitivity of the biomarker panel. The data, which are detailed below, shows that the biomarkers are associated with PMDD and PMD when examined in the luteal phase of the menstrual cycle, but surprisingly only in women whose symptoms are responsive to selective serotonin reuptake inhibitors (SSRI). Thus, the biomarkers can be used to identify individuals who are susceptible to depressive episodes that are triggered by a range of hormonal changes in women, and at the same time can be used to determine the most effective treatments for such individuals opening the door to the first precision medical treatments for these disorders.

### I. Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

As used herein, the term “comparing” refers to making an assessment of how the methylation status, proportion, level or cellular localization of one or more biomarkers in a sample from a patient relates to the methylation status, proportion, level or cellular localization of the corresponding one or more biomarkers in a standard or control sample. For example, “comparing” may refer to assessing whether the methylation status, proportion, level, or cellular localization of one or more biomarkers in a sample from a patient is the same as, more or less than, or different from the methylation status, proportion, level, or cellular localization of the corresponding one or more biomarkers in standard or control sample. More specifically, the term may refer to assessing whether the methylation status, proportion, level, or cellular localization of one or more biomarkers in a sample from a patient is the same as, more or less than, different from or otherwise corresponds (or not) to the methylation status, proportion, level, or cellular localization of predefined biomarker levels that correspond to, for example, a patient having PMDD or PMD, at risk for developing PMDD or PMD, not having PMDD or PMD, is responding to treatment for PMDD or PMD, is not responding to treatment for PMDD or PMD, is/is not likely to respond to a particular PMDD or PMD treatment, or having/not having another disease or condition. In a specific embodiment, the term “comparing” refers to assessing whether the methylation level of one or more biomarkers of the present invention in a sample from a patient is the same as, more or less than, different from other otherwise correspond (or not) to methylation levels of the same biomarkers in a control sample (e.g., predefined levels that correlate to uninfected individuals, standard PMDD lor PMD levels, etc.). As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has PMDD or PMD. In specific embodiments, the parameter may comprise the methylation status or level of one or more biomarkers of the present invention. A particular set or pattern of methylation of one or more biomarkers may indicate that a patient has PMDD or PMD (i.e., correlates to a patient having PMDD or PMD) or is at risk of developing PMDD or PMD. In other embodiments, a particular set or pattern of methylation of one or more biomarkers may be correlated to a patient being unaffected. In certain embodiments, “indicating,” or “correlating,” as used according to the present invention, may be by any linear or non-linear method of quantifying the relationship between methylation levels of biomarkers to a standard, control or comparative value for the assessment of the diagnosis, prediction of PMDD or PMDD progression, assessment of efficacy of clinical treatment, identification of a patient that may respond to a particular treatment regime or pharmaceutical agent, monitoring of the progress of treatment, and in the context of a screening assay, for the identification of an anti-PMDD therapeutic.

The term “Hormone replacement therapy” or “HRT”, refers to a form of hormone therapy used to treat symptoms associated with insufficient naturally circulating hormones, and is typically used to treat symptoms of hormone deficiency in older men and women in andropause or menopause. In women these symptoms can include hot flashes, vaginal atrophy, accelerated skin aging, vaginal dryness, decreased muscle mass, sexual dysfunction, and bone loss or osteoporosis. HRT typically includes one or more of the five major human steroid hormones: estrogens, progestogens, androgens, mineralocorticoids, and glucocorticoids. Estrogens and progestogens are the two most often used in menopause. They are available in a wide variety of FDA approved and non-FDA-approved formulations. In women with intact uteruses, estrogens are almost always given in combination with progestogens, as long-term unopposed estrogen therapy is associated with a markedly increased risk of endometrial hyperplasia and endometrial cancer. Conversely, in women who have undergone a hysterectomy or do not have a uterus, a progestogen is not required, and estrogen can be used alone. There are many combined formulations which include both estrogen and progestogen. Specific types of hormone replacement include for example: estrogens such bioidentical estrogens like estradiol and estriol, animal-derived estrogens like conjugated estrogens (CEEs), and synthetic estrogens like ethinylestradiol; progestogens such as bioidentical progesterone, and progestins (synthetic progestogens) like medroxyprogesterone acetate (MPA), norethisterone, and dydrogesterone and androgens such bioidentical testosterone and dehydroepiandrosterone (DHEA), and synthetic anabolic steroids like methyltestosterone and nandrolone decanoate. Dosage is often varied cyclically to more closely mimic the ovarian hormone cycle, with estrogens taken daily and progestogens taken for about two weeks every month or every other month, a schedule referred to as ‘cyclic’ or ‘sequentially combined’. Alternatively, ‘continuous combined’ HRT can be given with a constant daily hormonal dosage. Continuous combined HRT is associated with less complex endometrial hyperplasia than cyclic.

The terms “patient,” “individual,” or “subject” are used interchangeably herein, and refer to a mammal, particularly, a human. The patient may have mild, intermediate or severe disease, or may only experience transient, or fluctuating symptoms. The patient may be an individual, at risk of developing a disease, in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters, and primates. In one aspect the patient is a women of reproductive age (typically 13-40). In some aspects the patient is an older women (40-65) at risk of perimenopausal depression. In some aspects the women is a trans-man (i.e. a patient born as a women who has a gender identity more closely associated with a man, who is undergoing, or undergone transition to become a trans-man). In some aspects, the women is a trans-women (i.e. a patient born as a man who has a gender identity more closely associated with a woman, who is undergoing, or undergone transition to become a trans-women).

The term “PMDD” or “Premenstrual dysphoric disorder” (DSM-5 625.4 (N94.3)) refers to a combination of symptoms that begin in the final week before menses, started to improve in the days after onset of menses and are absent in the postmenstrual weeks during the past year. At least one of 5 or more required symptoms must be marked lability of affect, including irritability or anger or increased interpersonal conflict, depressed mood or hopelessness or self-deprecation, or marked anxiety or tension. Decreased interest in usual activities, subjective difficulty in concentrating, lethargy or fatigue or lack of energy, marked appetite change with overeating or food cravings, insomnia or hypersomnia, feelings of being out of control and somatic symptoms such as bloating, weight gain, breast tenderness, and joint or muscle pain may also be present (American Psychiatrica Association, 2013). As many as 80 percent of the women in the United States experience premenstrual emotional or physical symptoms (Boyle, Berkowitz & Kelsey, 1987). Between 3 and 8 percent of women generally meet the criteria for PMDD, with a wide range according to geography and perhaps culture, from 3 percent of women in Switzerland and 6 percent in India to 36 percent of female medical students in Nigeria) Symptoms occur principally during the late luteal phase of the menstrual cycle but in the first 2 or 3 days of the follicular phase in about one-third. Symptoms last on average 6 days and are most intense just before and after the start of menstrual flow. Feelings of sadness and despair, even including suicidality, anxiety and panic attacks, crying, irritability and anger, lack of interest in or attention to activities and relationships, fatigue and tiredness, difficulty focusing or thinking, food cravings and binge eating, and feeling out of control are common. Assorted somatic symptoms include breast tenderness, bloating, headache and pain. These symptoms resolve after the onset of menstruation

The term “Perimenopausal Depression” or “PMD” (also known as Major Depressive Disorder with peripartum onset in the DSM-5) refers to a subtype of depression experienced by women during the perimenopausal period, defined as the interval when a women's menstrual cycles become irregular, usually between ages of 45 and 49. The transition to menopause (or perimenopause, the beginning of ovarian failure) begins when menstrual cycles become 7 days longer or shorter than usual and extends to the early postmenopausal years. Menopause is defined as 12 months of amenorrhea following the final menstrual cycle, which usually occurs at an average age of 51 years.

The terms “measuring” and “determining” are used interchangeably throughout and refer to methods which include obtaining a patient sample and/or detecting the methylation status or level of a biomarker(s) in a sample. In one embodiment, the terms refer to obtaining a patient sample and detecting the methylation status or level of one or more biomarkers in the sample. In another embodiment, the terms “measuring” and “determining” mean detecting the methylation status or level of one or more biomarkers in a patient sample. Measuring can be accomplished by methods known in the art and those further described herein including, but not limited to, quantitative polymerase chain reaction (PCR). The term “measuring” is also used interchangeably throughout with the term “detecting.”

The term “methylation” refers to cytosine methylation at positions C5 or N4 of cytosine, the N6 position of adenine or other types of nucleic acid methylation. In vitro amplified DNA is unmethylated because in vitro DNA amplification methods do not retain the methylation pattern of the amplification template. However, “unmethylated DNA” or “methylated DNA” can also refer to amplified DNA whose original template was unmethylated or methylated, respectively. By “hypermethylation” or “elevated level of methylation” is meant an increase in methylation of a region of DNA (e.g., a biomarker of the present invention) that is considered statistically significant over levels of a control population. “Hypermethylation” or “elevated level of methylation” may refer to increased levels seen in a patient over time.

In particular embodiments, a biomarker would be unmethylated in a normal sample (e.g., normal or control tissue without disease, or normal or control body fluid, stool, blood, serum, amniotic fluid), in healthy stool, blood, serum, amniotic fluid or other body fluid. In other embodiments, a biomarker would be hypermethylated in a sample from a patient having or at risk of PMDD or PMD, such as at a methylation frequency of at least about 50%, at least about 60%, at least about 70%, at least about 75%, at least about 80%, at least about 85%, at least about 90%, at least about 95%, or about 100%.

A “methylation profile” refers to a set of data representing the methylation states or levels of one or more loci within a molecule of DNA from e.g., the genome of an individual or cells or sample from an individual. The profile can indicate the methylation state of every base in an individual, can comprise information regarding a subset of the base pairs (e.g., the methylation state of specific restriction enzyme recognition sequence) in a genome, or can comprise information regarding regional methylation density of each locus. In some embodiments, a methylation profile refers to the methylation states or levels of one or more biomarkers described herein, including HP1BP3 and TTC9B. In more specific embodiments, a methylation profile refers to the methylation states or levels of the promoter regions of HP1BP3 and TTC9B. In even more specific embodiments, a methylation profile refers to the methylation states of levels of CpG dinucleotides located within the region chr1:20986708-20986650 (human genome build hg18) and/or CpG dinucleotides located at chr19:45416573 (human genome build hg18). In some aspects a methylation profile refers to the methylation states or levels of the human MS4A gene cluster on Chromosome 11q12.

The terms “methylation status” or “methylation level” refers to the presence, absence and/or quantity of methylation at a particular nucleotide, or nucleotides within a portion of DNA. The methylation status of a particular DNA sequence (e.g., a DNA biomarker or DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value” or “methylation level.” A methylation value or level can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above-described example, represents the methylation status and can thus be used as a quantitative indicator of methylation status. This is of particular use when it is desirable to compare the methylation status of a sequence in a sample to a threshold value.

A “methylation-dependent restriction enzyme” refers to a restriction enzyme that cleaves or digests DNA at or in proximity to a methylated recognition sequence but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC). For example, McrBC's recognition sequence is 5′ RmC (N40-3000) RmC 3′ where “R” is a purine and “mC” is a methylated cytosine and “N40-3000” indicates the distance between the two RmC half sites for which a restriction event has been observed. McrBC generally cuts close to one half-site or the other, but cleavage positions are typically distributed over several base pairs, approximately 30 base pairs from the methylated base. McrBC sometimes cuts 3′ of both half sites, sometimes 5′ of both half sites, and sometimes between the two sites. Exemplary methylation-dependent restriction enzymes include, e.g., McrBC, McrA, MrrA, BisI, GlaI and DpnI. One of skill in the art will appreciate that any methylation-dependent restriction enzyme, including homologs and orthologs of the restriction enzymes described herein, is also suitable for use in the present invention.

A “methylation-sensitive restriction enzyme” refers to a restriction enzyme that cleaves DNA at or in proximity to an unmethylated recognition sequence but does not cleave at or in proximity to the same sequence when the recognition sequence is methylated. Exemplary methylation-sensitive restriction enzymes are described in, e.g., McClelland et al., 22(17) NUCLEIC ACIDS RES. 3640-59 (1994) and http://rebase.neb.com. Suitable methylation-sensitive restriction enzymes that do not cleave DNA at or near their recognition sequence when a cytosine within the recognition sequence is methylated at position C5 include, e.g., Aat II, Aci I, Acd I, Age I, Alu I, Asc I, Ase I, AsiS I, Bbe I, BsaA I, BsaH I, BsiE I, BsiW I, BsrF I, BssH II, BssK I, BstB I, BstN I, BstU I, Cla I, Eae I, Eag I, Fau I, Fse I, Hha I, HinP1 I, HinC II, Hpa II, Hpy99 I, HpyCH4 IV, Kas I, Mbo I, Mlu I, MapA1 I, Msp I, Nae I, Nar I, Not I, Pm1 I, Pst I, Pvu I, Rsr II, Sac II, Sap I, Sau3A I, Sfl I, Sfo I, SgrA I, Sma I, SnaB I, Tsc I, Xma I, and Zra I. Suitable methylation-sensitive restriction enzymes that do not cleave DNA at or near their recognition sequence when an adenosine within the recognition sequence is methylated at position N6 include, e.g., Mbo I. One of skill in the art will appreciate that any methylation-sensitive restriction enzyme, including homologs and orthologs of the restriction enzymes described herein, is also suitable for use in the present invention. One of skill in the art will further appreciate that a methylation-sensitive restriction enzyme that fails to cut in the presence of methylation of a cytosine at or near its recognition sequence may be insensitive to the presence of methylation of an adenosine at or near its recognition sequence. Likewise, a methylation-sensitive restriction enzyme that fails to cut in the presence of methylation of an adenosine at or near its recognition sequence may be insensitive to the presence of methylation of a cytosine at or near its recognition sequence. For example, Sau3 AI is sensitive (i.e., fails to cut) to the presence of a methylated cytosine at or near its recognition sequence, but is insensitive (i.e., cuts) to the presence of a methylated adenosine at or near its recognition sequence. One of skill in the art will also appreciate that some methylation-sensitive restriction enzymes are blocked by methylation of bases on one or both strands of DNA encompassing of their recognition sequence, while other methylation-sensitive restriction enzymes are blocked only by methylation on both strands but can cut if a recognition site is hemi-methylated.

The term “MS4A” refers to the human MS4A gene cluster on Chromosome 11q12 (see e.g., Liang et al., Structural organization of the human MS4A gene cluster on Chromosome 11q12 (Immunogenetics 2001) July; 53(5):357-68 doi: 10.1007/s002510100339). All MS4A family members share common structural features and similar intron/exon splice boundaries and are clustered along an approximately 600-kb region of Chromosome 11q The MS4A gene cluster encodes a family of proteins spanning the cellular membrane four times which share similar polypeptide sequence and predicted topological structure. Methylation loci within the MS4A7 cluster associated CpG sites include for example, cg14632030, cg08716584, cg09372819, cg18343292, cg10853416, cg20761290, cg25608560 and cg07048326.

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of PMDD and/or perimenopausal depression. Moreover, a sample obtained from a patient can be divided and only a portion may be used for diagnosis. Further, the sample, or a portion thereof, can be stored under conditions to maintain sample for later analysis. The definition specifically encompasses blood and saliva and other liquid samples of biological origin (including, but not limited to, peripheral blood, serum, plasma, urine, saliva, amniotic fluid, stool and synovial fluid), solid tissue samples such as a biopsy specimen or tissue cultures or cells derived therefrom and the progeny thereof. In a specific embodiment, a sample comprises a blood sample. In a specific embodiment, a sample comprises a saliva sample. Samples may be collected as part of routine physician visits, e.g., at the doctor's office, or via home collection kits, e.g. at home. In another embodiment, a serum sample is used. In another embodiment, a sample comprises amniotic fluid. In yet another embodiment, a sample comprises amniotic fluid. The definition also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

The term “SSRI” refers to Selective Serotonin Reuptake Inhibitor. SSRI antidepressants act by increasing levels of serotonin within the brain by preventing the reuptake of serotonin by nerves. Although all SSRI antidepressants are thought to act in the same way, there are differences between individual SSRIs with regards to how long they remain in the body, how they are metabolized, and how much they interact with other medications. For example, fluoxetine, fluvoxamine, and paroxetine are more likely to interact with other medications than citalopram, escitalopram and sertraline. Exemplary SSRIs include for example, Lexapro (Generic name: escitalopram), Zoloft (Generic name: sertraline), Prozac (Generic name: fluoxetine), Paxil (Generic name: paroxetine), Celexa (Generic name: citalopram), Luvox (Generic name: fluvoxamine), Paxil CR (Generic name: paroxetine), Brisdelle (Generic name: paroxetine), Sarafem (Generic name: fluoxetine), Luvox CR (Generic name: fluvoxamine), Prozac Weekly (Generic name: fluoxetine), Pexeva (Generic name: paroxetine), Selfemra (Generic name: fluoxetine) & Rapiflux (Generic name: fluoxetine).

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed for their methylation level in a sample from an unaffected individual (UI) or a normal control individual (NC) (both terms are used interchangeably herein). In another embodiment, a “suitable control” or “appropriate control” is a value, level, feature, characteristic, property, etc. determined prior to performing a therapy (e.g., a PMDD and/or perimenopausal depression treatment) on a patient. In yet another embodiment, a transcription rate, mRNA level, translation rate, protein level, biological activity, cellular characteristic or property, genotype, phenotype, etc. can be determined prior to, during, or after administering a therapy into a cell, organ, or patient. In a further embodiment, a “suitable control” or “appropriate control” is a predefined value, level, feature, characteristic, property, etc. A “suitable control” can be a methylation profile of one or more biomarkers of the present invention that correlates to PMDD and/or perimenopausal depression, to which a patient sample can be compared. The patient sample can also be compared to a negative control, i.e., a methylation profile that correlates to not having PMDD and/or perimenopausal depression.

### II. Hypermethylated Biomarkers and Detection Thereof

The biomarkers of the present invention are differentially methylated in PMDD and/or perimenopausal depression versus normal tissue. Such biomarkers can be used individually as diagnostic tool, or in combination as a biomarker panel. In some embodiments, the biomarkers include HP1BP3 and TTC9B In more specific embodiments, the biomarkers comprise the promoter regions of HP1BP3 and TTC9B. In even more specific embodiments, the biomarkers comprise CpG dinucleotides located within the region chr1:20986708-20986650 (human genome build hg18) (HP1BP3) and/or CpG dinucleotides located at chr19:45416573 (human genome build hg18) (TTC9B). The sequences of these biomarkers are publicly available.

In some embodiments, the biomarkers include HP1BP3 and TTC9B and MS4A. In more specific embodiments, the biomarkers comprise the promoter regions of HP1BP3 and TTC9B and MS4A7. In even more specific embodiments, the biomarkers comprise CpG dinucleotides located within the region chr1:20986708-20986650 (human genome build hg18) (HP1BP3) and/or CpG dinucleotides located at chr19:45416573 (human genome build hg18) (TTC9B), and/or CpG dinucleotides with the region of Chromosome 11q12. The sequences of these biomarkers are publicly available.

In some embodiments, the ratio of monocytes to non-monocytes can be assessed to improve biomarker model performance. The ratio of monocytes to non-monocytes can be determined by any known methods known in the art either by direct measurement or the use of proxies related to those cell types. These methods may include, for example, counting cell proportions as in through a differential CBC, measuring intermediate outputs of those cell types like cell surface antigens, or quantifying levels of cell type specific gene products like RNA expression or DNA methylation levels associated with the particular cell types. In one aspect, the ratio of monocytes to non-monocytes may be determined by measuring the DNA methylation status of the MS4A7 locus, a monocyte expressed gene which serves as a proxy for this ratio.

The DNA biomarkers of the present invention comprise fragments of a polynucleotide (e.g., regions of genome polynucleotide or DNA) which likely contain CpG island(s), or fragments which are more susceptible to methylation or demethylation than other regions of genome DNA. The term “CpG islands” is a region of genome DNA which shows higher frequency of 5′-CG-3′ (CpG) dinucleotides than other regions of genome DNA. Methylation of DNA at CpG dinucleotides, in particular, the addition of a methyl group to position 5 of the cytosine ring at CpG dinucleotides, is one of the epigenetic modifications in mammalian cells. CpG islands often harbor the promoters of genes and play a pivotal role in the control of gene expression. In normal tissues CpG islands are usually unmethylated, but a subset of islands becomes methylated during the development of a disease or condition (e.g., PMDD and/or perimenopausal depression).

There are a number of methods that can be employed to measure, detect, determine, identify, and characterize the methylation status/level of a biomarker (i.e., a region/fragment of DNA or a region/fragment of genome DNA (e.g., CpG island-containing region/fragment)) in the development of a disease or condition (e.g., PMDD and/or perimenopausal depression) and thus diagnose the onset, presence or status of the disease or condition.

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. See, e.g., U.S. Pat. No. 7,186,512. Alternatively, the method can encompass amplifying intact DNA after restriction enzyme digestion, thereby only amplifying DNA that was not cleaved by the restriction enzyme in the area amplified. See, e.g., U.S. Pat. Nos. 7,910,296; 7,901,880; and 7,459,274. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

In other embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

The quantity of methylation of a locus of DNA can be determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample. Such assays are disclosed in, e.g., U.S. Pat. No. 7,910,296.

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602, as well as in, e.g., DeGraves, et al., 34(1) Biotechniques 106-15 (2003); Deiman B, et al., 20(2) MOL. BIOTECHNOL. 163-79 (2002); and Gibson et al., 6 Genome Research 995-1001 (1996). Amplifications may be monitored in “real time.”

Additional methods for detecting DNA methylation can involve genomic sequencing before and after treatment of the DNA with bisulfite. See, e.g., Frommer et al., 89 Proc. Natl. Acad. Sci. USA 1827-31 (1992). When sodium bisulfite is contacted to DNA, unmethylated cytosine is converted to uracil, while methylated cytosine is not modified. In some embodiments, restriction enzyme digestion of PCR products amplified from bisulfite-converted DNA is used to detect DNA methylation. See, e.g., Xiong & Laird, 25 Nucleic Acids Res. 2532-34 (1997); and Sadri & Hornsby, 24 Nucl. Acids Res. 5058-59 (1996).

In some embodiments, a MethyLight assay is used alone or in combination with other methods to detect DNA methylation. See, Eads et al., 59 Cancer Res. 2302-06 (1999). Briefly, in the MethyLight process genomic DNA is converted in a sodium bisulfite reaction (the bisulfite process converts unmethylated cytosine residues to uracil). Amplification of a DNA sequence of interest is then performed using PCR primers that hybridize to CpG dinucleotides. By using primers that hybridize only to sequences resulting from bisulfite conversion of unmethylated DNA, (or alternatively to methylated sequences that are not converted) amplification can indicate methylation status of sequences where the primers hybridize. Similarly, the amplification product can be detected with a probe that specifically binds to a sequence resulting from bisulfite treatment of a unmethylated (or methylated) DNA. If desired, both primers and probes can be used to detect methylation status. Thus, kits for use with MethyLight can include sodium bisulfite as well as primers or detectably labeled probes (including but not limited to Taqman or molecular beacon probes) that distinguish between methylated and unmethylated DNA that have been treated with bisulfite. Other kit components can include, e.g., reagents necessary for amplification of DNA including but not limited to, PCR buffers, deoxynucleotides, and a thermostable polymerase.

In other embodiments, a Methylation-sensitive Single Nucleotide Primer Extension (Ms-SNuPE) reaction is used alone or in combination with other methods to detect DNA methylation. See Gonzalgo & Jones, 25 Nucleic Acids Res. 2529-31 (1997). The Ms-SNuPE technique is a quantitative method for assessing methylation differences at specific CpG sites based on bisulfite treatment of DNA, followed by single-nucleotide primer extension. Briefly, genomic DNA is reacted with sodium bisulfite to convert unmethylated cytosine to uracil while leaving 5-methylcytosine unchanged. Amplification of the desired target sequence is then performed using PCR primers specific for bisulfate-converted DNA, and the resulting product is isolated and used as a template for methylation analysis at the CpG site(s) of interest. Typical reagents (e.g., as might be found in a typical Ms-SNuPE-based kit) for Ms-SNuPE analysis can include, but are not limited to: PCR primers for specific gene (or methylation-altered DNA sequence or CpG island); optimized PCR buffers and deoxynucleotides; gel extraction kit; positive control primers; Ms-SNuPE primers for a specific gene; reaction buffer (for the Ms-SNuPE reaction); and detectably-labeled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

In further embodiments, a methylation-specific PCR reaction is used alone or in combination with other methods to detect DNA methylation. A methylation-specific PCR assay entails initial modification of DNA by sodium bisulfite, converting all unmethylated, but not methylated, cytosines to uracil, and subsequent amplification with primers specific for methylated versus unmethylated DNA. See, Herman et al., 93 Proc. Natl. Acad. Sci. USA 9821-26, (1996); and U.S. Pat. No. 5,786,146.

Additional methylation detection methods include, but are not limited to, methylated CpG island amplification (see, Toyota et al., 59 Cancer Res. 2307-12 (1999)) and those methods described in, e.g., U.S. Pat. Nos. 7,553,627; 6,331,393; U.S. patent Ser. No. 12/476,981; U.S. Patent Publication No. 2005/0069879; Rein, et al., 26(10) Nucleic Acids Res. 2255-64 (1998); and Olek et al., 17(3) Nat. Genet. 275-6 (1997).

In some embodiments DNA methylation detection is performed with the Illumina Infinium Methylation Assay (or similar commercially available instruments & technology) using a custom designed chip which uses ‘BeadChip’ technology to generate a selective analysis of human DNA methylation patterns. Similar to bisulfite sequencing and pyrosequencing, this method quantifies methylation levels at various loci within the genome. The processing and analysis of the Illumina Infinium assay is summarized below:

### Bisulfite Treatment

Approximately 1 μg of genomic DNA is used in bisulfite conversion to convert the unmethylated cytosine into uracil. The product contains unconverted cytosine where they were previously methylated, but cytosine is converted to uracil if they were previously unmethylated.

### Whole-Genomic DNA Amplification

The bisulfite treated DNA is subjected to whole-genome multiple displacement amplification via random hexamer priming and Φ29 DNA polymerase, which has a proofreading activity resulting in error rates 100 times lower than the Taq polymerase. The products are then enzymatically fragmented, purified from dNTPs, primers and enzymes, and applied to the chip.

### Hybridization and Single-Base Extension

On the chip, there are two bead types for each CpG site per locus. Each locus tested is differentiated by different bead types. Both bead types are attached to single-stranded 50-mer DNA oligonucleotides that differ in sequence only at the free end; this type of probe is known as an allele-specific oligonucleotide. One of the bead types will correspond to the methylated cytosine locus and the other will correspond to the unmethylated cytosine locus, which has been converted into uracil during bisulfite treatment and later amplified as thymine during whole-genome amplification. The bisulfite-converted amplified DNA products are denatured into single strands and hybridized to the chip via allele-specific annealing to either the methylation-specific probe or the non-methylation probe. Hybridization is followed by single base extension with hapten-labeled dideoxynucleotides. The ddCTP and ddGTP are labeled with biotin while ddATP and ddUTP are labeled with 2,4-dinitrophenol (DNP).

### Fluorescence Staining and Scanning of Chip

After incorporation of these hapten-labeled ddNTPs, multilayered immunohistochemical assays are performed by repeated rounds of staining with a combination of antibodies to differentiate the two types. After staining, the chip is scanned to show the intensities of the unmethylated and methylated bead types. The raw data are analyzed by the proprietary software, and the fluorescence intensity ratios between the two bead types are calculated. For a given individual at a given locus, a ratio value of 0 equals to non-methylation of the locus (i.e., homozygous unmethylated); a ratio of 1 equals to total methylation (i.e., homozygous methylated); and a value of 0.5 means that one copy is methylated and the other is not (i.e., heterozygosity), in the diploid human genome.

### Analysis of Methylation Data

The scanned microarray images of methylation data are further analyzed by the system, which normalizes the raw data to reduce the effects of experimental variation, background and average normalization, and performs standard statistical tests on the results. The data can then be compiled into several types of figures for visualization and analysis.

### Custom Chip Validation Studies

Prior to using the custom chip in clinical studies, the chip is validated at a CLIA certified vendor by running 25 duplicate samples run on the custom methylation array which are paired with at least 25 samples from the EPIC 850k array to provide the necessary data for validation.

### III. Determination of a Patient's PMDD and/or Perimenopausal Depression PMD Status

The present invention relates to the use of biomarkers to detect or predict PMDD and/or perimenopausal depression. More specifically, the biomarkers of the present invention can be used in diagnostic tests to determine, qualify, and/or assess PMDD and/or perimenopausal depression status, for example, to diagnose or predict PMDD and/or perimenopausal depression, in an individual, subject or patient. More specifically, the biomarkers to be detected in diagnosing PMDD and/or perimenopausal depression include, but are not limited to, HP1BP3 and TTC9B. Other biomarkers known in the relevant art may be used in combination with the biomarkers described herein, or example as proxy markers to assess the ratio of monocytes to non-monocytes. In one aspect the additional biomarker loci is MS4A. In one more specific aspect the additional biomarker loci is MS4A7.

### A. Biomarker Panels

The biomarkers of the present invention can be used in diagnostic tests to assess, determine, confirm and/or qualify (used interchangeably herein) PMDD and/or perimenopausal depression status in a patient. The phrase “PMDD status” or “perimenopausal depression status” includes any distinguishable transient or permanent manifestations of the disease, as well unaffected patients. For example, PMDD status includes, without limitation, the presence or absence of PMDD in a patient), the risk of developing PMDD, the stage of PMDD, the progress of PMDD (e.g., progress of PMDD over time) and the effectiveness or response to treatment of PMDD (e.g., clinical follow up and surveillance of PMDD after treatment), and SSRI responder status. Based on this status, further procedures may be indicated, including additional diagnostic tests or therapeutic procedures or regimens.

Similarly, PMD status includes, without limitation, the presence or absence of PMD in a patient), the risk of developing PMD, the stage of PMD, the progress of PMD (e.g., progress of PMD over time) and the effectiveness or response to treatment of PMD (e.g., clinical follow up and surveillance of PMD after treatment), and SSRI responder status. Based on this status, further procedures may be indicated, including additional diagnostic tests or therapeutic procedures or regimens

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the biomarker panels of the present invention may show a statistical difference in different PMDD and/or perimenopausal depression statuses of at least p<0.05, p<10-2, p<10-3, p<10-4 or p<10-5. Diagnostic tests that use these biomarkers may show an ROC of at least 0.6, at least about 0.7, at least about 0.8, or at least about 0.9.

The biomarkers are differentially methylated in UI (or NC) and PMDD and/or perimenopausal depression, and, therefore, are useful in aiding in the determination of PMDD and/or perimenopausal depression status. In certain embodiments, the biomarkers are measured in a patient sample using the methods described herein and compared, for example, to predefined biomarker levels and correlated to PMDD and/or perimenopausal depression status. In particular embodiments, the measurement(s) may then be compared with a relevant diagnostic amount(s), cut-off(s), or multivariate model scores that distinguish a positive PMDD and/or perimenopausal depression status from a negative PMDD and/or perimenopausal depression status. The diagnostic amount(s) represents a measured amount of a hypermethylated biomarker(s) above which or below which a patient is classified as having a particular PMDD and/or perimenopausal depression status. For example, if the biomarker(s) is/are hypermethylated compared to normal during PPD and/or perimenopausal depression, then a measured amount(s) above the diagnostic cutoff(s) provides a diagnosis of PMDD and/or perimenopausal depression. Alternatively, if the biomarker(s) is/are hypomethylated in a patient, then a measured amount(s) at or below the diagnostic cutoff(s) provides a diagnosis of non-PMDD and/or non-perimenopausal depression. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarker hypermethylation in a statistically significant number of samples from patients with the different PMDD and/or perimenopausal depression statuses and drawing the cut-off to suit the desired levels of specificity and sensitivity.

Indeed, as the skilled artisan will appreciate there are many ways to use the measurements of the methylation status of two or more biomarkers in order to improve the diagnostic question under investigation. In a quite simple, but nonetheless often effective approach, a positive result is assumed if a sample is hypermethylation positive for at least one of the markers investigated.

Furthermore, in certain embodiments, the methylation values measured for markers of a biomarker panel are mathematically combined and the combined value is correlated to the underlying diagnostic question. Methylated biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating methylation status of a biomarker combination of the present invention, e.g. to diagnose PMDD, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. Of Computational And Graphical Statistics 475-511 (2003); Friedman, J. H., 84 J. Of The American Statistical Association 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 Machine Learning 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

B. Determining Risk of Developing PMDD and/or Perimenopausal Depression

In a specific embodiment, the present invention provides methods for determining the risk of developing PMDD and/or perimenopausal depression in a patient. Biomarker methylation percentages, amounts or patterns are characteristic of various risk states, e.g., high, medium or low. The risk of developing PMDD and/or perimenopausal depression is determined by measuring the methylation status of the relevant biomarkers and then either submitting them to a classification algorithm or comparing them with a reference amount, i.e., a predefined level or pattern of methylated (and/or unmethylated) biomarkers that is associated with the particular risk level.

C. Determining PMDD and/or Perimenopausal Depression Severity

In another embodiment, the present invention provides methods for determining the severity of PMDD and/or perimenopausal depression in a patient. A particular stage or severity of PMDD and/or perimenopausal depression may have a characteristic level of hypermethylation of a biomarker or relative hypermethylated levels of a set of biomarkers (a pattern). The severity of PMDD and/or perimenopausal depression can be determined by measuring the methylation status of the relevant biomarkers and then either submitting them to a classification algorithm or comparing them with a reference amount, i.e., a predefined methylation level or pattern of methylated biomarkers that is associated with the particular stage.

D. Determining PMDD and/or Perimenopausal Depression Prognosis

In one embodiment, the present invention provides methods for determining the course of PMDD and/or perimenopausal depression in a patient. PMDD and/or perimenopausal depression course refers to changes in PMDD and/or perimenopausal depression status over time, including PMDD and/or perimenopausal depression progression (worsening) and PMDD and/or perimenopausal depression regression (improvement). Over time, the amount or relative amount (e.g., the pattern) of hypermethylation of the biomarkers changes. For example, hypermethylation of biomarker “X” and “Y” may be increased with PMDD and/or perimenopausal depression. Therefore, the trend of these biomarkers, either increased or decreased methylation over time toward PMDD and/or perimenopausal depression or non-PMDD and/or non-perimenopausal depression indicates the course of the disease. Accordingly, this method involves measuring the methylation level or status of one or more biomarkers in a patient at least two different time points, e.g., a first time and a second time, and comparing the change, if any. The course of PMDD and/or perimenopausal depression is determined based on these comparisons.

### E. Determining SSRI Responsivity

One aspect provides a method to select a PMDD or PMD patient that will respond to SSRI therapy in subjects which have already or are currently diagnosed with either mood disorder. SSRI responsiveness is determined by measuring the methylation status of the relevant biomarkers and then either submitting them to a classification algorithm or comparing them with a reference amount, i.e., a predefined level or pattern of methylated (and/or unmethylated) biomarkers that is associated with SSRI responsiveness.

### F. PMDD & PMD Therapies

In certain embodiments of the methods of qualifying PMDD and/or perimenopausal depression status, the methods further comprise administering the most appropriate PMDD therapy or PMD therapy. Such therapies include the actions of the physician or clinician subsequent to determining PMDD and/or perimenopausal depression status. For example, if a physician makes a diagnosis or prognosis of PMDD and/or perimenopausal depression, then a certain regime of monitoring would follow. An assessment of the course of PMDD and/or perimenopausal depression using the methods of the present invention may then require a certain PMDD and/or perimenopausal depression therapy regimens. In some aspects the biomarker panels can be used to determine SSRI responsiveness, and then this information can be used by the physician to prescribe the appropriate type of anti-depressant for use in a PMDD or PMD therapy. In some aspects the biomarker panels can be used to determine whether a patient is a candidate for HRT, and/or anti-depressants therapy. In some aspects the biomarker panels can be used to determine the type of HRT most suitable for the patient, such as continuous or cyclic HRT dosing regimens as known in the art. Alternatively, a diagnosis of non-PMDD and/or non-perimenopausal depression might be followed with further testing to determine a specific disease that the patient might be suffering from. Also, further tests may be called for if the diagnostic test gives an inconclusive result on PMDD and/or perimenopausal depression status.

### G. Determining Therapeutic Efficacy

In another embodiment, the present invention provides methods for determining the therapeutic efficacy of a pharmaceutical drug for treating, PMDD or PMD. These methods are useful in performing clinical trials of the drug, as well as monitoring the progress of a patient on the drug, or example to determine whether the patient is responding to the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of hypermethylation of one or more of the biomarkers of the present invention may change toward a non-PMDD and/or non-perimenopausal depression profile. Therefore, one can follow the course of the methylation status of one or more biomarkers in the patient during the course of treatment. Accordingly, this method involves measuring methylation levels of one or more biomarkers in a patient receiving drug therapy and correlating the levels with the PMDD and/or perimenopausal depression status of the patient (e.g., by comparison to predefined methylation levels of the biomarkers that correspond to different PMDD and/or perimenopausal depression statuses). One embodiment of this method involves determining the methylation levels of one or more biomarkers at least two different time points during a course of drug therapy, e.g., a first time and a second time, and comparing the change in methylation levels of the biomarkers, if any. For example, the methylation levels of one or more biomarkers can be measured before and after drug administration or at two different time points during drug administration. The effect of therapy is determined based on these comparisons. If a treatment is effective, then the methylation status of one or more biomarkers will trend toward normal, while if treatment is ineffective, the methylation status of one or more biomarkers will trend toward PMDD and/or perimenopausal depression indications. Exemplary therapeutics include for example antidepressants, and allopregnanolone derivatives and analogues which act as GABA modulators and which have shown efficacy for PPD. Exemplary allopregnanolone derivatives include for example, brexanolone and zuranolone made by Sage Therapeutics. 20)

### H. Generation of Classification Algorithms for Qualifying PMDD and PMD Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application No. 2002/0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”), U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows® or Linux™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including R, C, C++, visual basic, etc.

The learning algorithms described above are useful both for developing classification algorithms for the biomarker biomarkers already discovered, and for finding new biomarker biomarkers. The classification algorithms, in turn, form the base for diagnostic tests by providing diagnostic values (e.g., cut-off points) for biomarkers used singly or in combination. H. Kits for the Detection of PMDD and/or Perimenopausal Depression Biomarkers

In another aspect, the present invention provides kits for qualifying PMDD and/or perimenopausal depression status, which kits are used to detect or measure the methylation status/levels of the biomarkers described herein. Such kits can comprise at least one polynucleotide that hybridizes to at least one of the diagnostic biomarker sequences of the present invention and at least one reagent for detection of gene methylation. Reagents for detection of methylation include, e.g., sodium bisulfate, polynucleotides designed to hybridize to a sequence that is the product of a biomarker sequence of the invention if the biomarker sequence is not methylated (e.g., containing at least one C→U conversion), and/or a methylation-sensitive or methylation-dependent restriction enzyme. The kits can further provide solid supports in the form of an assay apparatus that is adapted to use in the assay. The kits may further comprise detectable labels, optionally linked to a polynucleotide, e.g., a probe, in the kit. Other materials useful in the performance of the assays can also be included in the kits, including test tubes, transfer pipettes, and the like. The kits can also include written instructions for the use of one or more of these reagents in any of the assays described herein.

In some embodiments, the kits of the invention comprise one or more (e.g., 1, 2, 3, 4, or more) different polynucleotides (e.g., primers and/or probes) capable of specifically amplifying at least a portion of a DNA region of a biomarker of the present invention including HP1BP3 and TTC9B. Optionally, one or more detectably-labeled polypeptides capable of hybridizing to the amplified portion can also be included in the kit. In some embodiments, the kits comprise sufficient primers to amplify 2, 3, 4, 5, 6, 7, 8, 9, 10, or more different DNA regions or portions thereof, and optionally include detectably labeled polynucleotides capable of hybridizing to each amplified DNA region or portion thereof. The kits further can comprise a methylation-dependent or methylation sensitive restriction enzyme and/or sodium bisulfite.

In some embodiments, the kits comprise sodium bisulfite, primers and adapters (e.g., oligonucleotides that can be ligated or otherwise linked to genomic fragments) for whole genome amplification, and polynucleotides (e.g., detectably labeled polynucleotides) to quantify the presence of the converted methylated and or the converted unmethylated sequence of at least one cytosine from a DNA region of a biomarker of the present invention including HP1BP3 and TTC9B.

In some embodiments, the kits comprise a microarray comprising, consisting or, or consisting essentially of primers specific for HP1BP3 and TTC9B and MS4A. In more specific embodiments, the kits comprise oligonucleotide sequences which are specific the promoter regions of HP1BP3 and TTC9B and MS4A7. In even more specific embodiments, the kits comprise oligonucleotide sequences which are specific for CpG dinucleotides located within the region chr1:20986708-20986650 (human genome build hg18) (HP1BP3) and/or CpG dinucleotides located at chr19:45416573 (human genome build hg18) (TTC9B), and/or CpG dinucleotides with the region of Chromosome 11q12. The sequences of these biomarkers are publicly available.

In some embodiments, the kits comprise methylation sensing restriction enzymes (e.g., a methylation-dependent restriction enzyme and/or a methylation-sensitive restriction enzyme), primers and adapters for whole genome amplification, and polynucleotides to quantify the number of copies of at least a portion of a DNA region of a biomarker of the present invention including HP1BP3, TTC9B and MS4A.

In some embodiments, the kits comprise a methylation binding moiety and one or more polynucleotides to quantify the number of copies of at least a portion of a DNA region of a biomarker of the present invention including HP1BP3, TTC9B and MS4A. A methylation binding moiety refers to a molecule (e.g., a polypeptide) that specifically binds to methylcytosine. Examples include restriction enzymes or fragments thereof that lack DNA cutting activity but retain the ability to bind methylated DNA, antibodies that specifically bind to methylated DNA, etc.).

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example I Identification of Epigenetic Biomarkers for the Clinical Diagnosis of PMDD

### Introduction

Premenstrual dysphoric disorder (PMDD) is reproductive subtype of depression (McEvoy et al. 2017) that affects approximately five percent of menstruating individuals (Gehlert et al. 2009). PMDD's signature is a cluster of affective symptoms including irritability, low mood, affective lability, and anxiety, that occur during the luteal (premenstrual) phase of the menstrual cycle, but remit in the follicular phase (Epperson et al. 2012; Hantsoo and Epperson 2015). This roughly monthly emergence of mood symptoms causes significant impairment and distress for those with PMDD (Pearlstein and Steiner 2008). PMDD's pathogenesis is poorly understood, but like other reproductive affective disorders, likely involves altered central nervous system sensitivity to fluctuating neuroactive steroid hormones (Schmidt et al. 2017; Hantsoo and Epperson 2020).

In addition to PMDD's poorly characterized pathophysiology, little is known about what differentiates treatment responders from non-responders in PMDD with regard to the first-line treatment, selective serotonin reuptake inhibitors (SSRIs). SSRIs are often administered in a luteal phase dosing scheme, in which the SSRI is administered from ovulation until menses onset (Halbreich and Smoller 1997; Jermain et al. 1999; Miner et al. 2002; Halbreich et al. 2002; Freeman 2004; Freeman et al. 2005; Steiner et al. 2006, 2008; Eriksson et al. 2008). However, SSRIs are only effective in roughly 50-60% of individuals with PMDD (Dimmock et al. 2000; Halbreich et al. 2006; Halbreich 2008). This is an important gap in knowledge, as studies to date have not found factors such as demographics, menstrual cycle length, parity, nor premenstrual symptom profiles, that clearly differentiate PMDD SSRI responders from non-responders (Freeman et al. 1999, 2000). SSRIs act rapidly and at low doses in PMDD (Kornstein et al. 2006; Steinberg et al. 2012), suggesting a different mechanism of action than serotonergic pathways (Griffin and Mellon 1999; Pinna et al. 2009), potentially via regulating allopregnanolone levels (Gracia et al. 2009).

The present studies were aimed at assessing whether the previously identified epigenetic biomarkers predictive of PPD, a type of reproductive depression, might also be associated with PMDD, or perimenopausal depression.

### Material & Methods

### Participants

Female control and PMDD participants were recruited from the community via fliers, radio and television advertising, public transit advertisements and web-based clinical research recruitment portal (iConnect); advertisements were targeted separately toward controls and to women with premenstrual mood symptoms. Potential participants verbally consented to telephone screening to assess preliminary eligibility. Eligibility criteria included age 18-50 years old, experiencing regular menstrual cycles (24-39 days), English speaking and able to give written informed consent. Exclusion criteria included psychotropic medication use in the past 2 months; drug or alcohol abuse in the past 2 years; lifetime history of any psychotic disorder; history of Axis I disorder other than specific phobia in the past year; active suicidal ideation with plan or attempt in the past 6 months; steroid hormone or hormonal contraceptive use (except use of levonorgestrel as an emergency contraceptive) in the past 6 months; pregnancy in the past year.

If preliminarily eligible based on telephone screening, potential participants completed prospective daily rating of premenstrual symptoms with the Daily Record of Severity of Problems (DRSP) (Endicott et al. 2006) over two menstrual cycles to confirm absence of symptoms in controls, or sufficient premenstrual symptoms in the PMDD group. To be eligible for the control group, with the first day of menstrual flow designated as day 1, a participant could not have affective symptoms in the follicular or luteal phase with greater than mild severity (mean DRSP symptom scores≤3) nor with cyclicity (considering days 5-11 and days −7 to −1 of menstrual cycle). For the PMDD group, women could not have greater than mild affective mean symptom scores (≤3) in the follicular phase, and required at least a 50% increase in mean DRSP score for at least five of eleven symptoms, comparing the mean score for each symptom from the follicular phase (day 5 to 11) with the mean symptom score from the luteal phase (days −7 to −1), with at least one of the symptoms being irritability, mood lability, depressed mood, and/or anxiety/tension. These criteria are consistent with DSM-5 criteria for PMDD diagnosis (Epperson et al. 2012; American Psychiatric Assn 2013) Laboratory Protocol

Participants completed laboratory sessions in the follicular phase and the luteal phase. Order of the laboratory sessions was counterbalanced between participants and groups. Follicular sessions occurred during days 5 to 11 of the menstrual cycle, and luteal sessions occurred in the week prior to menses, eight to twelve days post-ovulation. Participants used a lutenizing hormone (LH) urine test kit (One Step Ultra-Sensitive 20 mIU/mL, Aide Diagnostic Company, Shandong, China) to determine ovulation. In addition, luteal phase was confirmed by serum progesterone levels ≥3 ng/ml within 24 hours prior to the luteal laboratory session. When progesterone levels did not confirm luteal status, participants were rescheduled in a subsequent luteal phase. Serum estradiol and progesterone were assessed at each laboratory session to confirm menstrual phase status.

### Self-Report Measures

Daily Record of Severity of Problems (DRSP): The DRSP is a daily symptom rating tool that includes eleven criterion symptoms of premenstrual dysphoric disorder plus three impairment items, rated on a scale of 1 (“not at all”) to 6 (“extreme”) (Endicott et al. 2006). To determine initial eligibility, participants completed the DRSP across two menstrual cycles. Then, participants continued to rate symptoms daily throughout study participation to ensure that women with PMDD continued to meet DSM criteria for PMDD, and that controls continued to be asymptomatic. The DRSP was also used as an outcome measure to determine sertraline impact on premenstrual symptoms, and treatment response. As an outcome measure, DRSP scores were calculated for the follicular phase and luteal phase of each menstrual cycle. The score for each item was averaged across the follicular week (day 5 to 11) and the luteal week (day −1 to −7). The item means were then summed to create a total DRSP score for the follicular phase and luteal phase, ranging from 14 to 84. As an indicator of treatment response, a 30% or greater improvement in DRSP total score was required.

### Biomarker Measurement

Blood for genetic assays was drawn at the beginning of either the follicular or luteal laboratory session. Approximately 10 ml of blood was drawn into a BD Vacutainer Plus plastic K2EDTA tube (Becton, Dickinson and Company, Franklin Lakes, NJ). The tube was agitated by hand and immediately placed in a −80° C. freezer for storage. Bisulfite conversion was carried out using EZ DNA Methylation Gold Kit (Zymo Research) according to the manufacturer's instructions. Nested PCR amplifications were performed with a standard PCR protocol in 25 ml volume reactions containing 3-4 μl of sodium-bisulfite-treated DNA, 0.2 μM primers, and master mix containing Taq DNA polymerase (Sigma Aldrich). Primer sequences can be found in below. PCR amplicons were processed for pyrosequencing analysis according to the manufacturer's standard protocol (Qiagen) using a PyroMark MD system (QIAGEN) with Pyro Q-CpG 1.0.9 software (QIAGEN) for CpG methylation quantification.

Primer Sequences

| Gene | Primer Name | Primer Sequence (5′-3′) |
|---|---|---|
| HP1BP3 | HP1BP3_F_ | ATTTTTTTAAATTAGTTTTGAAGAGTTGTA |
| | out | (SEQ ID NO: 1) |
| | HP1BP3_R_ | CCTAAAAAAAAATCCACCAAAAAAAC |
| | out | (SEQ ID NO: 2) |
| | HP1BP3_F_ | TTTTTTTGTATGTGAGGATTAGGGAG |
| | in | (SEQ ID NO: 3) |
| | HP1BP3_R_ | biotin-CAATCCCTTCTCTTAACTAAATT |
| | in | TCC (SEQ ID NO: 4) |
| | HP1BP3_ | TTAAAAAAAGGTTTGTTTTTGAGTTG |
| | Pyro1 | (SEQ ID NO: 5) |
| TTC9B | TTC9B_F_ | GGGGGAAAGAGTAGGAAGATA |
| | out | (SEQ ID NO: 6) |
| | TTC9B_R_ | AAACTAATCTCAAACTTCTAACCTC |
| | out | (SEQ ID NO: 7) |
| | TTC9B_F_ | biotin-TATTTTTTTATTAGTGGTATGAT |
| | in | TTAGATAGT (SEQ ID NO: 8) |
| | TTC9B_R_ | CCTAAAAATAATATTATTATACCATATTAC |
| | in | TAAT (SEQ ID NO: 9) |
| | TTC9B_ | TTATTAGTGGTATGATTTAGATAGT |
| | Pyro1 | (SEQ ID NO: 10) |

### Serum Hormone Measurement

Blood was drawn at each laboratory session to measure serum progesterone and estradiol levels, and progesterone levels were measured <24 hours prior to luteal phase laboratory sessions to confirm luteal status. Approximately 3.5 ml blood was drawn into a serum separator (SST) tube with silica clot activator, polymer gel, silicone-coated interior (Becton, Dickinson and Company, Franklin Lakes, NJ). The tube was centrifuged for fifteen minutes at room temperature (approximately 21° C.), then transported at room temperature to a clinical laboratory on campus where serum progesterone and estradiol levels were assayed by electrochemiluminescence immunoassay (ECLIA) (Siemens Immulite 2000, Malvern, PA). For estradiol, the analytical range was 20-2000 μg/mL and sensitivity 15 μg/mL, and for progesterone the analytical range was 0.2-40 ng/ml and sensitivity: 0.1 ng/mL.

### Sertraline Treatment

Participants with PMDD received sertraline at a dose of 50 mg daily during the luteal phase. Medication was open-label and dispensed by the University of Pennsylvania Investigational Drug Service. Once ovulation was detected using a urine LH kit, the participant began sertraline treatment and continued taking sertraline daily until onset of their next menstrual period. Sertraline treatment response was defined as at least a 30% improvement in DRSP total score from untreated luteal to treated luteal phases (Evans et al. 1999; Eisenlohr-Moul et al. 2017).

### Statistical Analyses

Demographics were summarized by group (control vs. PMDD). Continuous variables were summarized with means (standard deviations) and differences were tested with two-sample t tests; categorical variables were summarized with frequencies (percentages) and differences were tested with Fisher's exact tests. Inspection of the data showed no strong deviations from normality or model assumptions. Significance was considered at the p<0.05 level. Statistical analyses were performed with R version 3.5.1

### Results

### Sample Characteristics

One hundred and seventeen potential participants (controls and PMDD) completed the screening process. Of these, seventeen were not eligible, seventeen declined to continue in the study, and six were lost to follow-up. Of the remaining participants, seventy-seven completed one laboratory session, sixty-five completed two laboratory sessions, and fifty-seven completed all three laboratory sessions. Blood samples for the genetic biomarker were available for fifty-five participants (n=26 control, n=29 PMDD).

### Demographic and Health Characteristics

Table 1 summarizes demographic and health data in all enrolled individuals with biomarker data. The control (n=26) and PMDD (n=29) groups were similar with respect to race, education level, marital status, income, employment status, and body mass index (BMI) (p's>0.05). Participants in the control group were significantly younger than the PMDD group (p=0.008).

TABLE 1
Participant characteristics by group
Table 1. For categorical variables, data is presented as n (%)
and p-values are for Fisher's exact tests. For continuous
variables, data is presented as Mean (SD) and p-values are
for independent sample T-tests.

| | Control | | PMDD | | |
|---|---|---|---|---|---|
| | N = 26 | | N = 29 | | p-value |
| Race | | | | | 0.12 |
| Caucasian | 12 | (48.0) | 19 | (67.9) | |
| Black/African American | 7 | (28.0) | 8 | (28.6) | |
| Asian | 4 | (16.0) | 0 | (0.0) | |
| Other | 2 | (8.0) | 1 | (3.6) | |
| Education | | | | | 0.149 |
| High School | 1 | (4.0) | 2 | (6.9) | |
| Some College | 3 | (12.0) | 9 | (31.0) | |
| College Graduate | 13 | (52.0) | 7 | (24.1) | |
| At least some graduate school | 8 | (32.0) | 11 | (37.9) | |
| Marital Status | | | | | 0.254 |
| Married, Domestic partner | 5 | (20.0) | 11 | (37.9) | |
| Single, Separated, Divorced | 20 | (76.9) | 18 | (62.1) | |
| Income | | | | | 0.645 |
| <$50,000 | 15 | (60.0) | 13 | (44.8) | |
| $50,000-$100,000 | 5 | (20.0) | 9 | (31.0) | |
| $101,000-$150,000 | 2 | (8.0) | 4 | (13.8) | |
| >$150,000 | 3 | (12.0) | 3 | (10.3) | |
| Employment Status | | | | | 0.535 |
| Unemployed | 1 | (4.0) | 2 | (6.9) | |
| Part-Time | 5 | (20.0) | 6 | (20.7) | |
| Full-Time | 14 | (56.0) | 19 | (65.5) | |
| Student | 5 | (20.0) | 2 | (6.9) | |
| Age (years) | 28.27 | (5.40) | 33.20 | (7.30) | 0.008 |

| | | | |
|---|---|---|---|
| BMI | 25.27 | 25.43 | 0.841 |

### Serum Steroid Hormone Levels

Serum estradiol and progesterone levels were similar between controls and PMDD participants in both the follicular and luteal phases (p's>0.05) (Table 2).

TABLE 2
Outcomes by group
Table 2. Data is presented as Mean (SD), and p-values are
for independent sample T-tests. Serum hormone levels were
measured in the follicular phase for n = 6 control, n = 17
PMDD, and in the luteal phase for n = 20 control, n = 12 PMDD.

| | Control | | PMDD | | p-value |
|---|---|---|---|---|---|
| DRSP | | | | | |
| Follicular | 14.37 | (0.55) | 18.79 | (6.48) | 0.119 |
| Luteal | 14.84 | (0.81) | 31.04 | (11.55) | <0.001 |
| Estradiol (pg/ml) | | | | | |
| Follicular | 77.87 | (50.5) | 75.00 | (35.12) | 0.879 |
| Luteal | 104.54 | (37.82) | 121.94 | (51.20) | 0.479 |
| Progesterone (ng/ml) | | | | | |
| Follicular | 0.41 | (0.14) | 0.32 | (0.2) | 0.356 |
| Luteal | 6.45 | (3.6) | 8.11 | (4.35) | 0.311 |

### Mood Ratings and Sertraline Treatment Response

Total DRSP score was significantly higher in the PMDD group than in the control group in both the follicular (p=0.003) and luteal (p<0.001) phases. Within the PMDD group, twenty-three participants completed sertraline treatment. On average, participants took sertraline for M=8.5 (1.4) days. Within the PMDD group, 10 participants (43.48%) were considered sertraline treatment responders, and 13 were non-responders. Overall, total DRSP score decreased in response to sertraline treatment by 25%, from M=33.1 (13.7) in the untreated luteal phase to M=24.8 (9.6) in the treated luteal phase, an improvement of 8 points.

### Biomarker Analysis

The biomarkers were assessed in fifty-five participants (n=26 control, 29 PMDD). In 23 participants, the biomarkers were assessed in the follicular phase (n=6 control, 17 PMDD), and in 32 participants the biomarker was assessed in the luteal phase (n=20 control, 12 PMDD). Using DNA methylation data generated by sodium bisulfite pyrosequencing at TTC9B and HP1BP3 in a cohort of N=56 women with and without PMDD, reproductive depression model (PPD model) predictions were generated using the established PPD biomarker linear model.

Generation of reproductive depression model output is achieved using the model: E(ReproDi)=a+(bHP1BP3+c TTC9B)×d MS4A7 Where: Reproductive Depression (ReproD) status for individual (i) is modeled as a function of an interaction of HP1BP3 and TTC9B DNA methylation with monocyte proportion proxy biomarker (MS4A7). The model was trained on the previously published Johns Hopkins Prospective Cohort against postpartum depression (PPD) status (Guintivano et al., 2013), and model guesses generated using the ‘predict’ function in R.

The model prediction was evaluated as a function of PMDD diagnostic status as well as selective serotonin reuptake inhibitor (SSRI) responsiveness separately in subsets of samples collected during the follicular phase (N=25) and luteal phase (N=31) of the menstrual cycle.

In follicular phase samples, the model failed to distinguish PMDD cases (N=13) from controls (N=9), generating an AUC of 0.5 (95% CI: 0.23-0.77). Similarly, a weak distinction between SSRI responders (N=8) from SSRI non-responders (N=5) among women with PMDD was observed, generating an AUC of 0.63 (95% CI: 0.29-0.96).

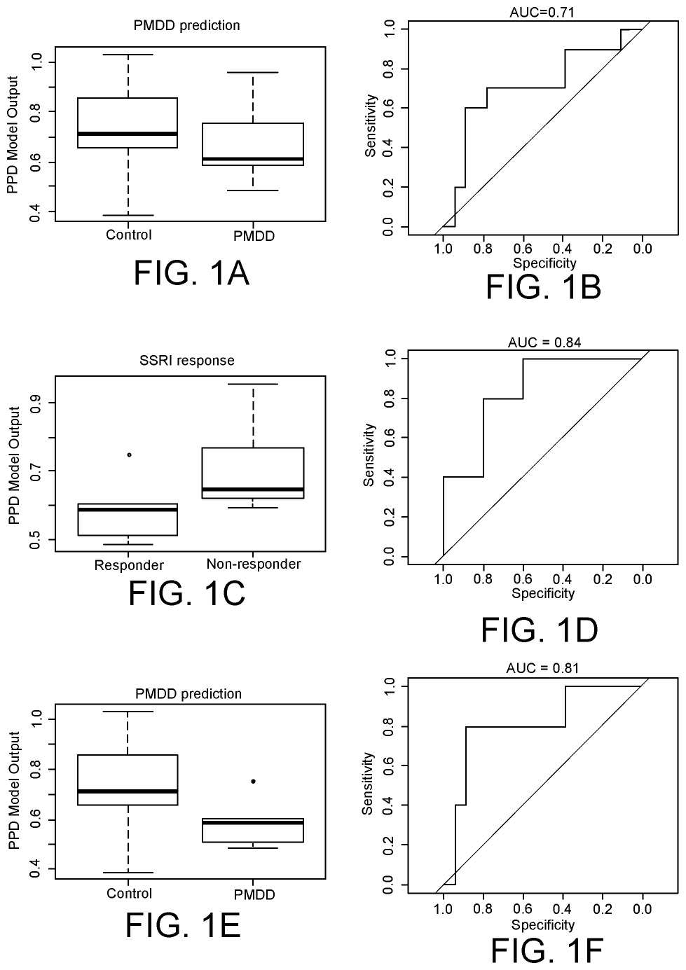

Surprisingly in view of the failure to observe any significant predictive ability of the biomarkers in the follicular phase, the luteal phase samples, the model performance was dramatically better generating an AUC of 0.71 (95% CI: 0.49-0.93) to distinguish N=10 PMDD cases from N=18 controls (FIG. 1A, B). The model predictions also generated an AUC of 0.84 (95% CI: 0.57-1) to distinguish between N=5 SSRI non-responders from N=5 SSRI responders (FIG. 1C, D). The direction of prediction between these two outcomes were in opposite directions, suggesting that the reproductive depression biomarker model applied in luteal phase, will predict PMDD from controls in women who are SSRI responders. To test this assertion, the N=5 SSRI non-responders were removed from the analysis and then it was attempted to predict PMDD status from N=5 PMDD and N=18 controls, generating an AUC of 0.81 (95% CI: 0.57-1) (FIG. 1E, F). In light of these results, it becomes possible to predict SSRI responder status from women with reproductive depression if we are aware of both the model output generated from the biological test and the diagnostic status of reproductive depression.

In summary, this work confirms that the previously identified epigenetic biomarkers extend to another type of reproductive depression, PMDD. Surprisingly the biomarkers are only effective when tested in the luteal phase of the menstrual cycle and strongly associated with SSRI-responsive PMDD. These findings indicate that the biomarkers are likely indicators of brain sensitivity to reproductive hormonal change in women. Critically these new findings enable new insights into the both the biological underpinnings of PMDD and its effective treatment and suggest that SSRI-responsive PMDD form of PMDD is likely a separate and distinct of PMDD from the SSRI-nonresponsive PMDD, and therefore should be therapeutically treated differentially.

To date, the clinical diagnosis of PMDD technically requires two months of prospective daily mood ratings in order to confirm the diagnosis and is unable to differentiate between the SSRI responsive and non-responsive forms. Clinically, we have now shown that the biomarkers can be used to quickly and unambiguously diagnose a subset of women with PMDD and predict response to SSRI treatment, thus eliminating the need for prospective monitoring. The identification of biomarkers for a subtype of PMDD, which is responsive to SSRI treatment, is therefore important advance in the field and it will allow one the first precision medical diagnosis for this important illness.

### Example 2 Validation of Epigenetic Biomarkers for Determining SSRI Responsiveness

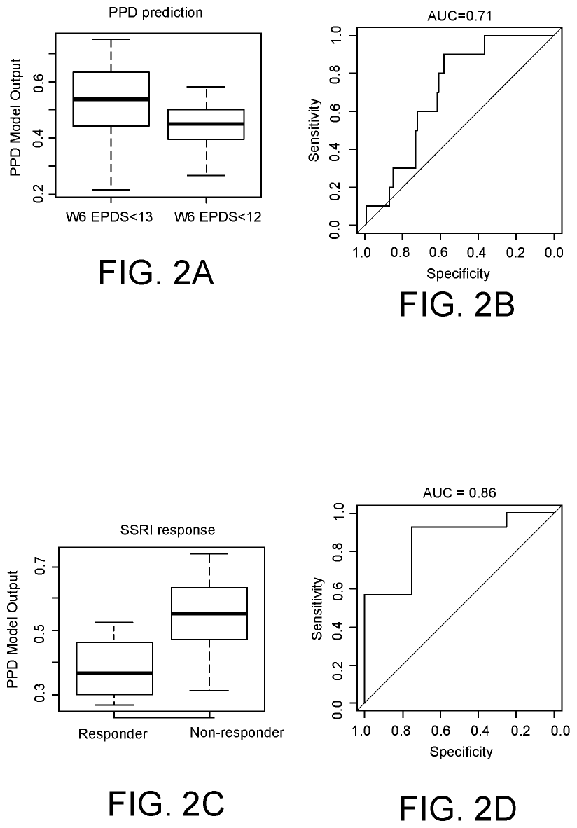

The World Health Organization, and CDC estimates that nearly 40 million people in Europe and about 17 million adult individuals in the US experiences depression every year¹⁹. It is estimated that 3 out of 4 people suffering from depression currently do not receive proper treatment. Accordingly, the development of improved methods to identify anti-depressant responsivity to enable the most effective treatments for depression are urgently needed. To validate our initial findings of SSRI responsiveness we used an alternative data set of DNA methylation data in women who were part of a longitudinal DNA methylation study of postpartum depression. Application of the SSRI response model to this third trimester data at TTC9B and HP1BP3 predicted N=10 women with Edinburgh Postnatal Depression Scale (EPDS) scores ≥13 at 6 weeks postpartum (W6) from N=108 women with EPDS <13 with an AUC of 0.71 (95% CI: 0.58-0.83) and a direction of model output prediction for cases relative to controls consistent with that observed in the PMDD cohort (FIG. 2). A metric of inferred SSRI treatment response was generated by looking at the change in EPDS symptoms between the W6 time point and at 3 months postpartum (M3). The rationale for these time periods is that most women will receive standard postpartum screening for depressive symptoms at W6, suggesting that this will be the period where depressive symptoms are most likely to be identified in a general practice setting and the point at which pharmacological treatment decisions will be made. Women on SSRIs for whom EPDS symptoms decreased by at least 3 scale points over the period were labeled as SSRI responders (N=4), while those for whom the change in EPDS scores over the period were above this value were labeled as SSRI non-responders (N=14). We then assessed the predictive accuracy and direction of SSRI response in this group, identified an AUC of SSRI response prediction of 0.86 (95% CI: 0.6372-1) and observed a pattern consistent with that observed in the PMDD cohort (FIG. 2).

### Example 3. Demonstration of the Ability of the Epigenetic Biomarkers to Predict the Development of Perimenopausal Depression (PMD)

Approximately 1.3 million women year enter menopause in the US. Perimenopausal depression is experienced by about 18% among women in early perimenopause and 38% of those in late perimenopause (Gao, M., Zhang, H., Gao, Z., Sun, Y., Wang, J., Wei, F. and Gao, D. Global hotspots and prospects of perimenopausal depression: A bibliometric analysis via CiteSpace Front. Psychiatry, 10 Sep. 2022 Sec. Aging Psychiatry https://doi.org/10.3389/fpsyt.2022.968629). Women with PMD report significantly decreased quality of life, social support, adjustment, suicidal thoughts, and increased disability compared to non-depressed individuals. The development of Perimenopausal depression increases a women's risk of developing Alzheimer's disease by 2-fold (Cherbuin N, Kim S, Anstey KJ. Dementia risk estimates associated with measures of depression: a systematic review and meta-analysis. BMJ Open. 2015; 5(12):e008853. Epub 2015 Dec. 23. https://doi.org/10.1136/bmjopen-2015-008853 PMID: 26692556; PubMed Central PMCID: PMC4691713).

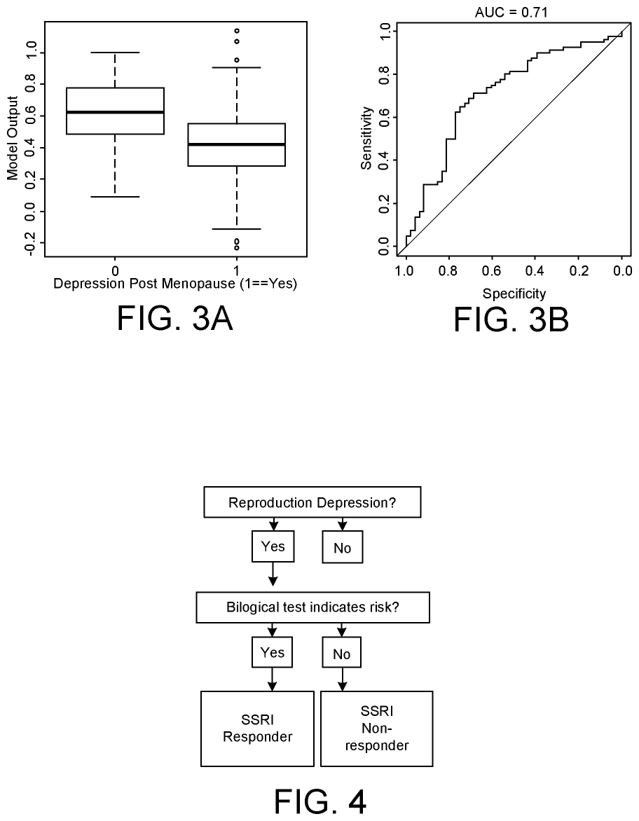

To evaluate whether we could extend the utility of the biomarkers to other reproductive depressions we assessed whether they were able to predict perimenopausal depression. To do this we assessed TTC9B and HP1BP3 DNA methylation levels in a sample of women older than 55 years old from the Gene Expression Omnibus (GSE125105). Of these samples, N=40 had a diagnosis of depression, while N=128 did not. The model generated a prediction AUC of 0.71 (95% CI: 0.6195-0.8091) (FIG. 3). The results demonstrate for the first time that TTC9B and HP1BP3 DNA methylation status can also be used to predict the future development of perimenopausal depression.

### CONCLUSIONS

These studies sought to examine whether the previously identified epigenetic biomarkers for PPD were also associated with other types of reproductive depression, including

PMDD and PMD. It was found that the epigenetic biomarkers were associated with PMDD but only during the luteal phase of the menstrual cycle, the phase of the cycle during which hormone levels fluctuate and trigger PMDD symptoms. Further, it was found that the model was more strongly predictive of PMDD in patients that were responsive to SSRI treatment. This finding supports previous work that suggested that at least some “reproductive depressions” might be uniquely and rapidly responsive to SSRIs and suggests that there are at least two biological subtypes of PMDD-those that are responsive and those that are nonresponsive to SSRIs.

The work also indicates that the epigenetic biomarkers are not just biomarkers of reproductive depressions, but are, also, biomarkers of brain sensitivity to reproductive hormonal change.

### BIBLIOGRAPHY

American Psychiatric Assn A (2013) Diagnostic and statistical manual of mental disorders (5th ed.), 5th edn. American Psychiatric Publishing, Arlington VA
Comasco E, Hahn A, Ganger S, et al (2014) Emotional fronto-cingulate cortex activation and brain derived neurotrophic factor polymorphism in premenstrual dysphoric disorder. Hum Brain Mapp 35:4450-4458. https://doi.org/10.1002/hbm.22486
Dhingra V, Magnay J L, O'Brien PMS, et al (2007) Serotonin receptor 1A C (−1019) G polymorphism associated with premenstrual dysphoric disorder. Obstet Gynecol 110:788-792. https://doi.org/10.1097/01.AOG.0000284448.73490.ac
Dimmock P W, Wyatt K M, Jones P W, O'Brien P M (2000) Efficacy of selective serotonin-reuptake inhibitors in premenstrual syndrome: a systematic review. Lancet Lond Engl 356:1131-1136. https://doi.org/10.1016/s0140-6736(00)02754-9
Dubey N, Hoffman J F, Schuebel K, et al (2017) The ESC/E(Z) complex, an effector of response to ovarian steroids, manifests an intrinsic difference in cells from women with premenstrual dysphoric disorder. Mol Psychiatry 22:1172-1184. https://doi.org/10.1038/mp.2016.229
Eisenlohr-Moul T A, Girdler S S, Schmalenberger K M, et al (2017) Toward the Reliable Diagnosis of DSM-5 Premenstrual Dysphoric Disorder: The Carolina Premenstrual Assessment Scoring System (C-PASS). Am J Psychiatry 174:51-59. https://doi.org/10.1176/appi.ajp.2016.15121510
Endicott J, Nee J, Harrison W (2006) Daily Record of Severity of Problems (DRSP): reliability and validity. Arch Womens Ment Health 9:41-49. https://doi.org/10.1007/s00737-005-0103-y
Epperson C N, Steiner M, Hartlage S A, et al (2012) Premenstrual dysphoric disorder: evidence for a new category for DSM-5. Am J Psychiatry 169:465-475
Eriksson E, Ekman A, Sinclair S, et al (2008) Escitalopram administered in the luteal phase exerts a marked and dose-dependent effect in premenstrual dysphoric disorder. J Clin Psychopharmacol 28:195-202. https://doi.org/10.1097/JCP.0b013e3181678a28
Evans S M, Foltin R W, Fischman M W (1999) Food “cravings” and the acute effects of alprazolam on food intake in women with premenstrual dysphoric disorder. Appetite 32:331-349. https://doi.org/10.1006/appe.1998.0222
Freeman E W (2004) Luteal phase administration of agents for the treatment of premenstrual dysphoric disorder. CNS Drugs 18:453-468
Freeman E W, Rickels K, Sondheimer S J, Polansky M (1999) Differential response to antidepressants in women with premenstrual syndrome/premenstrual dysphoric disorder: a randomized controlled trial. Arch Gen Psychiatry 56:932-939. https://doi.org/10.1001/archpsyc.56.10.932
Freeman E W, Sondheimer S J, Polansky M, Garcia-Espagna B (2000) Predictors of response to sertraline treatment of severe premenstrual syndromes. J Clin Psychiatry 61:579-584. https://doi.org/10.4088/jcp.v61n0807
Freeman E W, Sondheimer S J, Sammel M D, et al (2005) A preliminary study of luteal phase versus symptom-onset dosing with escitalopram for premenstrual dysphoric disorder. J Clin Psychiatry 66:769-773
Gehlert S, Song I H, Chang C-H, Hartlage S A (2009) The prevalence of premenstrual dysphoric disorder in a randomly selected group of urban and rural women. Psychol Med 39:129-136. https://doi.org/10.1017/S003329170800322X
Gingnell M, Comasco E, Oreland L, et al (2010) Neuroticism-related personality traits are related to symptom severity in patients with premenstrual dysphoric disorder and to the serotonin transporter gene-linked polymorphism 5-HTTPLPR. Arch Womens Ment Health 13:417-423. https://doi.org/10.1007/s00737-010-0164-4
Gracia C R, Freeman E W, Sammel M D, et al (2009) Allopregnanolone levels before and after selective serotonin reuptake inhibitor treatment of premenstrual symptoms. J Clin Psychopharmacol 29:403-405. https://doi.org/10.1097/JCP.0b013e3181ad8825
Griffin L D, Mellon S H (1999) Selective serotonin reuptake inhibitors directly alter activity of neurosteroidogenic enzymes. Proc Natl Acad Sci USA 96:13512-13517
Guintivano J, Arad M, Gould T D, et al (2014) Antenatal prediction of postpartum depression with blood DNA methylation biomarkers. Mol Psychiatry 19:560-567. https://doi.org/10.1038/mp.2013.62
Halbreich U (2008) Selective serotonin reuptake inhibitors and initial oral contraceptives for the treatment of PMDD: effective but not enough. CNS Spectr 13:566-572
Halbreich U, Bergeron R, Yonkers K A, et al (2002) Efficacy of intermittent, luteal phase sertraline treatment of premenstrual dysphoric disorder. Obstet Gynecol 100:1219-1229
Halbreich U, O'Brien P M S, Eriksson E, et al (2006) Are there differential symptom profiles that improve in response to different pharmacological treatments of premenstrual syndrome/premenstrual dysphoric disorder? CNS Drugs 20:523-547
Halbreich U, Smoller J W (1997) Intermittent luteal phase sertraline treatment of dysphoric premenstrual syndrome. J Clin Psychiatry 58:399-402
Hantsoo L, Epperson C N (2015) Premenstrual Dysphoric Disorder: Epidemiology and Treatment. Curr Psychiatry Rep 17:87. https://doi.org/10.1007/s11920-015-0628-3
Hantsoo L, Epperson C N (2020) Allopregnanolone in premenstrual dysphoric disorder (PMDD): Evidence for dysregulated sensitivity to GABA-A receptor modulating neuroactive steroids across the menstrual cycle. Neurobiol Stress 12:100213. https://doi.org/10.1016/j.ynstr.2020.100213
Huo L, Straub R E, Roca C, et al (2007) Risk for premenstrual dysphoric disorder is associated with genetic variation in ESR1, the estrogen receptor alpha gene. Biol Psychiatry 62:925-933. https://doi.org/10.1016/j.biopsych.2006.12.019
Jermain D M, Preece C K, Sykes R L, et al (1999) Luteal phase sertraline treatment for premenstrual dysphoric disorder. Results of a double-blind, placebo-controlled, crossover study. Arch Fam Med 8:328-332
Kornstein S G, Pearlstein T B, Fayyad R, et al (2006) Low-dose sertraline in the treatment of moderate-to-severe premenstrual syndrome: efficacy of 3 dosing strategies. J Clin Psychiatry 67:1624-1632
Magnay J L, Ismail K M K, Chapman G, et al (2006) Serotonin transporter, tryptophan hydroxylase, and monoamine oxidase A gene polymorphisms in premenstrual dysphoric disorder. Am J Obstet Gynecol 195:1254-1259. https://doi.org/10.1016/j.ajog.2006.06.087
Marrocco J, Einhorn N R, Petty G H, et al (2020) Epigenetic intersection of BDNF Val66Met genotype with premenstrual dysphoric disorder transcriptome in a cross-species model of estradiol add-back. Mol Psychiatry 25:572-583. https://doi.org/10.1038/s41380-018-0274-3
McEvoy K, Osborne L M, Nanavati J, Payne J L (2017) Reproductive Affective Disorders: a Review of the Genetic Evidence for Premenstrual Dysphoric Disorder and Postpartum Depression. Curr Psychiatry Rep 19:94. https://doi.org/10.1007/s11920-017-0852-0
Melke J, Westberg L, Landén M, et al (2003) Serotonin transporter gene polymorphisms and platelet [3H] paroxetine binding in premenstrual dysphoria. Psychoneuroendocrinology 28:446-458. https://doi.org/10.1016/s0306-4530(02)00033-1
Miner C, Brown E, McCray S, et al (2002) Weekly luteal-phase dosing with enteric-coated fluoxetine 90 mg in premenstrual dysphoric disorder: a randomized, double-blind, placebo-controlled clinical trial. Clin Ther 24:417-433. https://doi.org/10.1016/s0149-2918(02)85043-3
Osborne L, Clive M, Kimmel M, et al (2016) Replication of Epigenetic Postpartum Depression Biomarkers and Variation with Hormone Levels. Neuropsychopharmacology 41:1648-1658. https://doi.org/10.1038/npp.2015.333
Payne J L, Osborne L M, Cox O, et al (2020) DNA methylation biomarkers prospectively predict both antenatal and postpartum depression. Psychiatry Res 285:112711. https://doi.org/10.1016/j.psychres.2019.112711
Pearlstein T, Steiner M (2008) Premenstrual dysphoric disorder: burden of illness and treatment update. J Psychiatry Neurosci JPN 33:291-301
Pinna G, Costa E, Guidotti A (2009) SSRIs act as selective brain steroidogenic stimulants (SBSSs) at low doses that are inactive on 5-HT reuptake. Curr Opin Pharmacol 9:24-30. https://doi.org/10.1016/j.coph.2008.12.006
Schmidt P J, Martinez P E, Nieman L K, et al (2017) Premenstrual Dysphoric Disorder Symptoms Following Ovarian Suppression: Triggered by Change in Ovarian Steroid Levels But Not Continuous Stable Levels. Am J Psychiatry 174:980-989. https://doi.org/10.1176/appi.ajp.2017.16101113
Steinberg E M, Cardoso G M P, Martinez P E, et al (2012) Rapid response to fluoxetine in women with h premenstrual dysphoric disorder. Depress Anxiety 29:531-540. https://doi.org/10.1002/da.21959
Steiner M, Pearlstein T, Cohen L S, et al (2006) Expert guidelines for the treatment of severe PMS, PMDD, and comorbidities: the role of SSRIs. J Womens Health 2002 15:57-69. https://doi.org/10.1089/jwh.2006.15.57
Steiner M, Ravindran A V, LeMelledo J-M, et al (2008) Luteal Phase Administration of Paroxetine for the Treatment of Premenstrual Dysphoric Disorder: A Randomized, Double-Blind, Placebo-Controlled Trial in Canadian Women. J Clin Psychiatry 69:991-998
Ullah A, Long X, Mat W-K, et al (2020) Highly Recurrent Copy Number Variations in GABRB2 Associated With Schizophrenia and Premenstrual Dysphoric Disorder. Front Psychiatry 11:572. https://doi.org/10.3389/fpsyt.2020.00572
Yen J-Y, Wang P-W, Su C-H, et al (2018) Estrogen levels, emotion regulation, and emotional symptoms of women with premenstrual dysphoric disorder: The moderating effect of estrogen receptor 1α polymorphism. Prog Neuropsychopharmacol Biol Psychiatry 82:216-223. https://doi.org/10.1016/j.pnpbp.2017.11.013

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

1-4. (canceled)

5. A method to diagnose a patient having one or more symptoms of premenstrual dysphoric disorder (PMDD) and/or at risk of perimenopausal depression (PMD) comprising measuring DNA methylation levels of a panel of biomarker loci in a nucleic acid sample obtained from the patient, wherein differential DNA methylation levels of the panel of biomarker loci relative to a corresponding panel of biomarker loci from a patient not having PMDD and/or PMD is indicative of the patient having a diagnosis of PMDD and/or PMD, wherein the panel of biomarker loci comprises CpG dinucleotides in HP1BP3 loci (chr1:20986708-20986650 of human genome build hg 18) and TTC9B loci (chr19:45416573 of human genome build hg 18).

6. The method of claim 5, wherein the panel of biomarker loci further comprises at least one additional DNA methylation biomarker as a proxy marker to estimate the ratio monocytes:non-monocytes in the sample.

7. The method of claim 6, wherein the additional DNA methylation biomarker loci is MS4A.

8-14. (canceled)

15. The method of claim 5, wherein the patient is a naturally cycling woman.

16. The method of claim 15, wherein the sample is collected during the patient's luteal phase.

17-18. (canceled)

19. The method of any one of claim 5,
wherein the HP1BP3 loci comprises CpG dinucleotides located within the minus strand of chr1:20986708-20986650 of human genome build hg 18 or
wherein the TTC9B loci comprises CpG dinucleotides located within the plus strand of chr19:45416573 of human genome build hg 18.

20-22. (canceled)

23. The method of claim 6, wherein the methylation levels of MS4A gene cluster loci are used as a proxy the ratio of monocytes:non-monocytes, wherein the MS4A loci comprises CpG dinucleotides located within the human MS4A gene cluster on Chromosome 11q12.

24-27. (canceled)

28. The method of claim 5, wherein the DNA methylation levels are measured after sodium bisulfite modification and analyzed using microarray analysis.

29. The method of claim 5, further comprising using a linear model that utilizes the DNA methylation level of HP1BP3 and TTC9B and the ratio of monocytes non-monocytes or proxy marker to determine the patient is at an increased risk of developing PMDD and/or perimenopausal depression, wherein the linear model utilizes DNA methylation at HP1BP3 interacting with the ratio of monocytes:non-monocytes or proxy marker and utilizes DNA methylation at TTC9B as an additive covariate.

30. The method of claim 23, wherein the linear model utilizes DNA methylation at HP1BP3 and TTC9B as additive covariates and the ratio of monocytes:non-monocytes or proxy marker as an interacting component.

31. The method of claim 5, further comprising using a linear model that utilizes the DNA methylation level of HP1BP3 and TTC9B and the ratio of monocytes:non-monocytes or proxy marker to determine the patient is at an increased risk of developing PMDD and/or perimenopausal depression, wherein the linear model uses methylation levels of MS4A gene cluster loci are used as a proxy the ratio of monocytes:non-monocytes.

32-40. (canceled)

41. A method to treat a patient responsive to serotonin reuptake inhibitors (SSRIs) and at risk of developing, or having, premenstrual dysphoric disorder (PMDD) and/or perimenopausal depression (PMD) comprising a) identifying the patient is an SSRI responder, and b) administering to the patient an effective SSRI,
wherein the risk of developing premenstrual dysphoric disorder (PMDD) and/or perimenopausal depression (PMD) and responsiveness to SSRIs is determined by:
i) measuring white blood cell type counts and determining a ratio of monocytes:non-monocytes in the sample collected from the patient during the patient's luteal phase or using a proxy marker to estimate the ratio monocytes:non-monocytes in the sample;
ii) measuring DNA methylation levels of a panel of biomarker loci in the sample collected from the patient, wherein the panel of biomarker loci comprises CpG dinucleotides in HP1BP3 loci (chr1:20986708-20986650 of human genome build hg 18) and TTC9B loci (chr19:45416573 of human genome build hg 18); and
iii) using a linear model that utilizes the DNA methylation level of HP1BP3 and TTC9B and the ratio of monocytes:non-monocytes or proxy marker to determine the patient is at an increased risk of developing PMDD and/or PMD and if the patient will be responsive to selective serotonin reuptake inhibitors (SSRIs).

42. The method of claim 41, wherein methylation levels of MS4A gene cluster loci are used as a proxy the ratio of monocytes:non-monocytes.

43. The method of claim 42, wherein the MS4A loci comprises CpG dinucleotides located within the human MS4A gene cluster on Chromosome 11q12, wherein the MSA4 loci is MS4A7.

44-45. (canceled)

46. A method to treat a patient at risk of developing premenstrual dysphoric disorder (PMDD) and/or perimenopausal depression (PMD) comprising administering to the patient an effective anti-depressant, wherein the risk of developing premenstrual dysphoric disorder (PMDD) and/or perimenopausal depression (PMD) is determined by:
a) measuring white blood cell type counts and determining a ratio of monocytes:non-monocytes in the sample collected from the patient during the patient's luteal phase or using a proxy marker to estimate the ratio monocytes:non-monocytes in the sample;
b) measuring DNA methylation levels of a panel of biomarker loci in the sample collected from the patient, wherein the panel of biomarker loci comprises CpG dinucleotides in HP1BP3 loci (chr1:20986708-20986650 of human genome build hg 18) and TTC9B loci (chr19:45416573 of human genome build hg 18); and
c) using a linear model that utilizes the DNA methylation level of HP1BP3 and TTC9B and the ratio of monocytes:non-monocytes or proxy marker to determine the patient is at an increased risk of developing PMDD and/or PMD.

47. The method of claim 46, wherein the methylation levels of MS4A gene cluster loci are used as a proxy the ratio of monocytes:non-monocytes.

48. The method of claim 47, wherein the MS4A loci comprises CpG dinucleotides located within the human MS4A gene cluster on Chromosome 11q12, wherein the MSA4 loci is MS4A7.

49-54. (canceled)

55. The method of claim 41,
wherein the HP1BP3 loci comprises CpG dinucleotides located within the minus strand of chr1:20986708-20986650 of human genome build hg 18,
wherein the TTC9B loci comprises CpG dinucleotides located within the plus strand of chr19:45416573 of human genome build hg 18.

56-59. (canceled)

60. The method of claim 41, wherein the methylation levels of MS4A gene cluster loci are used as a proxy the ratio of monocytes:non-monocytes, wherein the MS4A loci comprises CpG dinucleotides located within the human MS4A gene cluster on Chromosome 11g12, wherein the loci is MS4A7.

61-64. (canceled)

65. The method of claim 41, DNA methylation levels are measured after sodium bisulfite modification and analyzed using microarray analysis.

66. The method of claim 41, wherein the linear model utilizes DNA methylation at HP1BP3 interacting with the ratio of monocytes:non-monocytes or a proxy marker for the ratio monocytes:non-monocytes in the sample, and utilizes DNA methylation at TTC9B as an additive covariate.

67. The method of claim 41, wherein linear model utilizes DNA methylation at HP1BP3 and TTC9B as additive covariates and the ratio of monocytes:non-monocytes or a proxy marker for the ratio monocytes:non-monocytes in the sample, as an interacting component.

68. The method of claim 41, wherein the linear model uses methylation levels of MS4A gene cluster loci as a proxy the ratio of monocytes:non-monocytes.

69. The method of claim 68, wherein MS4A loci comprises CpG dinucleotides located within the human MS4A gene cluster on Chromosome 11q12, wherein the MS4A loci is MS4A7.

70-73. (canceled)

74. The method of claim 55, comprising administering PMMD therapy and/or PMD therapy to the patient.

75. The method of claim 74, wherein the PMMD therapy and/or PMD therapy is determined using a classification algorithm to determine PMMD and/or PMD status using methylation levels from known samples.

76. The method of claim 75, wherein the classification algorithm uses one or more additives or interactive covariates to determine the PMMD and/or PMD therapy of the patient.

77. The method of claim 75, wherein the classification algorithm uses stress, anxiety, or sleep quality metrics from the patient as an additive or interactive covariate.

78. The method of claim 77, wherein the stress, anxiety, or sleep quality metrics are taken from Pittsburgh Sleep Quality Scale (PSQI), Clinical Global Impression Scale (CGIS), Perceived Stress Scale (PSS) or a combination thereof.

79. The method of claim 74, wherein the PMDD therapy or PMD therapy is psychiatric therapy, Cognitive Behavioral Therapy (CBT), antidepressant therapy with a medication, hormone replacement therapy or a combination thereof.

80. The method of claim 79, wherein the antidepressant medication is a selective serotonin reuptake inhibitor (SSRI).

81. The method of claim 80, wherein the SSRI is citalopram, escitalopram, fluoxetine, fluvoxamine, paroxetine, sertraline, vilazodone or a combination thereof.

82-91. (canceled)


SEQUENCE LISTING (10 sequences)

SEQ ID NO: 1 - DNA, 30 bp, synthetic construct
  source 1..30: mol_type = other DNA
atttttttaaattagttttgaagagttgta

SEQ ID NO: 2 - DNA, 26 bp, synthetic construct
  source 1..26: mol_type = other DNA
cctaaaaaaaaatccaccaaaaaaac

SEQ ID NO: 3 - DNA, 26 bp, synthetic construct
  source 1..26: mol_type = other DNA
tttttttgtatgtgaggattagggag

SEQ ID NO: 4 - DNA, 26 bp, synthetic construct
  source 1..26: mol_type = other DNA
caatcccttctcttaactaaatttcc

SEQ ID NO: 5 - DNA, 26 bp, synthetic construct
  source 1..26: mol_type = other DNA
ttaaaaaaaggtttgtttttgagttg

SEQ ID NO: 6 - DNA, 21 bp, synthetic construct
  source 1..21: mol_type = other DNA
gggggaaagagtaggaagata

SEQ ID NO: 7 - DNA, 25 bp, synthetic construct
  source 1..25: mol_type = other DNA
aaactaatctcaaacttctaacctc

SEQ ID NO: 8 - DNA, 32 bp, synthetic construct
  source 1..32: mol_type = other DNA
tatttttttattagtggtatgatttagatagt

SEQ ID NO: 9 - DNA, 34 bp, synthetic construct
  source 1..34: mol_type = other DNA
cctaaaaataatattattataccatattactaat

SEQ ID NO: 10 - DNA, 25 bp, synthetic construct
  source 1..25: mol_type = other DNA
ttattagtggtatgatttagatagt